(19) United States
(12) Patent Application Publication
(10) Pub. No.: US 2005/0209104 A1
(43) Pub. Date: Sep. 22, 2005

(54) Dinoflagellate karlotoxins, methods of isolation and uses thereof

(76) Inventors: Allen Place, Baltimore, MD (US); Jonathan R. Deeds, Laurel, MD (US)

Correspondence Address: Steven J Hultquist;Intellectual Property Technology Law, PO Box 14329, Research Triangle Park, NC, 27709, US

(21) Appl. No.: 10/525,711

(22) PCT Filed: Aug. 19, 2003
(86) PCT No.: PCT/US03/25840

§ 371 Date: May 23, 2005

Related U.S. Application Data
(60) Provisional application No. 60/404,468, filed on Aug. 19, 2002.

(51) Int. Cl. A61K 39/395; A01N 59/00; C07K 14/405

(52) U.S. Cl. 504/150; 424/130.1; 530/350; 530/387.1

(57) ABSTRACT

Disclosed are six dinoflagellate toxins isolated from Karlodinium micrum and methods of isolating said toxins. The toxins are useful in killing cells, such as in the killing of tumor cells in an animal. Antibodies against these toxins allow detection of the toxins and the dinoflagellate as well as a method of neutralizing the toxin.

### BACKGROUND OF THE INVENTION

1. Field of the Invention

The present invention related to dinoflagellate toxins, and more particularly, to specific dinoflagellate toxins from Karlodinium micrum, isolation and purification thereof, and uses of said toxins.

2. Background of the Related Art

Dinoflagellates are microscopic, usually single-celled organisms and are commonly regarded as algae. Dinoflagellates are capable of producing toxins that are harmful to marine life as well as to humans. Several toxins have been isolated from dinoflagellates, including saxitoxin produced by Protogotryaulax catanella and Gessnerium monilatum, and brevitoxin produced by the dinoflagellate Karenia brevis. All of these toxins are neurotoxins.

Karlodinium micrum (Syn. Gymnodinium galatheanum, Gymnodinium micrum, Gyrodinium galatheanum) is a common estuarine non-thecate dinoflagellate that can form blooms in aquaculture or natural systems (Li et al. 1996, 2000; Nielsen 1996; Glibert and Terlizzi 1999). Karlotoxins are ichthyotoxic and pose a problem to the aquaculture of fish. Blooms of K. micrum have been reported in association with fish kills (e.g. Braarud 1957; Larsen and Moestrup 1989; Nielsen 1996; Terlizzi et al. 2000; Lewitus et al. 2002; Deeds et al. 2002), and toxic effects of K. micrum cultures on mussels (Mytilus edulis) and juvenile cod (Gadus morhua) have been demonstrated (Nielsen and Strømgren 1991; Nielsen 1993).

While the dinoflagellate Karlodinium micrum has been associated with fish kills, heretofore no toxic substance has been identified in Karlodinium micrum. However, isolation and identification of such toxins would be beneficial to detection and inactivation of such toxins.

### BRIEF SUMMARY OF THE INVENTION

The present invention relates to Karlodinium micrum toxins, methods of isolating said toxins, methods of using said toxins, methods of detecting said toxins and methods of inactivating said toxins.

In one aspect, the present invention relates to six isolated karlotoxins from K. micrum that exhibit ichthyotoxic, cytotoxic and hemolytic activity.

In another aspect, the present invention relates to six karlotoxins, identified as KmTx 1-KmTx 6, having different retention times on a C₁₈ HPLC column, wherein the retention times for the different toxins range from about 17 minutes to about 24 minutes. Culture filtrates of K. micrum cells comprise at least two distinct fractions that co-elute with polar lipids. Preferably, the retention times consist of approximately 16 to 18 minutes and approximately 22 to 25 minutes, and more preferably, about 17 minutes or about 23 minutes.

In yet another aspect, the present invention relates to antibodies against the K. micrum toxins for detection, isolation, sequestering, and/or inactivation of said toxins. A preferred embodiment of the instant invention is an antibody, either polyclonal or monoclonal, which binds a Karlodinium micrum toxin, and more preferably, binds any one of the karlotoxins KmTx 1, KmTx 2, KmTx 3, KmTx 4, KmTx 5 or KmTx 6.

Still another aspect relates to an immunoconjugate comprising a Karlodinium micrum toxin linked to an antibody, wherein the toxin is any one of the karlotoxins KmTx 1, KmTx 2, KmTx 3, KmTx 4, KmTx 5 or KmTx 6. Preferably, the toxin is linked to an anti-tumor antibody and the immunoconjugate is included in a pharmaceutical composition.

In another aspect, the present invention relates to methods of treatment of blooms caused by K. micrum to reduce the mortality rate of fish exposed thereto, the method comprising the introduction of potassium permanganate as an algicidal with the exclusion of copper sulfate, thereby reducing the release of karlotoxins caused by cell disturbance and/or damage of the K. micrum cells by the copper sulfate. Although treatment with either algicidal copper or potassium permanganate causes lysis of K. micrum cells (>70%), toxic activity was released after treatment with copper and eliminated following treatment with potassium permanganate.

Still another aspect of the present invention relates to karlotoxins having a molecular mass of 1362 and 1344 daltons, (KmTx 1 and KmTx 2, respectively) determined by liquid chromatography/mass spectrometry.

A still further aspect relates to compounds and pharmaceutical compositions for delivery to tumor or cancer cells for effective killing of such cells. Preferably, the compositions comprise an effective amount of any of the compounds KmTx 1-KmTx 6 to kill or reduce growth of cancer cell. More preferably, the compositions comprises from about 500 to about 2000 ng ml⁻¹. Further, the karlotoxins described herein may be used as antibiotics against bacteria or fungi.

Another aspect of the present invention relates to a method of producing a toxin comprising the steps of: a) culturing Karlodinium micrum in a medium suitable for production of toxin; and b) isolating the toxin.

Other features and advantages of the invention will be apparent from the following detailed description, drawings and claims.

### BRIEF DESCRIPTION OF THE DRAWINGS

FIGS. 1A and B show reversed phase HPLC elution profiles at 230 nm for a 25 ul injection of a concentrated 80% MeOH tC₈ elution of Mount Pleasant retention pond filtrates (0.22 um) from (A) Feb. 5, 2002 (650 ml), and (B) Feb. 6, 2002 (1 liter). Flow rate of separation was 1 ml min⁻¹. Overlaid histogram (gray bars) is the hemolytic activity of cells lysed with 10 μg saponin, in 0.5 min-collected fractions.

FIGS. 2A and B show (A) co-injection of aliquots of hemolytic HPLC fractions from South Carolina Karlodinium micrum isolate 010410-C6, and Chesapeake Bay Karlodinium micrum isolate CCMP 1974, scanned at 230 nm (dashed line) and 254 nm (solid line). (B) UV spectra of hemolytic HPLC peak eluting at 23 min from Chesapeake Bay Karlodinium micrum isolate CCMP 1974 (solid line) and the hemolytic HPLC peak eluting at 22 min from a water sample collected during a Mount Pleasant, S.C. fish kill on 6 Feb. 2002 (dashed line) which contained 6.8×10⁴ Karlodinium micrum cells ml⁻¹.

FIG. 3 is a map of the Chesapeake Bay showing location of HyRock Fish Farm, positioned on the Manokin River in Princess Ann county, Md., USA.

FIGS. 4A, B, C and D illustrate the dose dependence for the lysis of rainbow trout erythrocytes, compared to cells lysed with 10 μg saponin, for (A) The standard hemolysin saponin, (B) A diluted suspension of sonicated Karlodinium micrum (CCMP 1974; 1.2×10⁵ cells ml⁻¹), (C) Reversed-phase HPLC fraction 46/47 (KmTx 1), elution time 23 min., (D) Reversed-phase HPLC fraction 36 (KmTx 3), elution time 17.5 min. (n=4).

FIGS. 5A, B, C and D show percent hemolysis, measured as release of hemoglobin compared to cells lysed with 10 μg saponin, in aliquots of separated lipid classes from Karlodinium micrum (CCMP 1974). (A) Grown autotrophically (=4.78×10⁷ cells ml⁻¹). (B) Grown autotrophically (=1.55×10⁷ cells ml⁻¹). (C) Grown mixotrophically (=1.0×10⁷ cells ml⁻¹). (D)) Cryptophyte food source Storeatula major (strain g) (=3.44×10⁷ cells ml⁻¹). According to Yongmanitchi and Ward (1992) fractions included: (i.) neutral lipids, (ii.) monogalactosyl-diacylglycerol (MGDG), (iii.) digalactosyl-diacylglycerol (DGDG), (iv.) sulfoquinovo-diacylglycerol (SQDG), (v.) unknown acyllipid, (vi.) phosphatidylcholine (PC), (vii.) lysophospholipid (LC). Control consisted of an equivalent amount of MeOH. Bars represent one standard deviation; n=4 (x) Signifies 100% mortality (15 of 15 larvae) occurred in 48 hour bioassay using zebrafish (Danio rerio). (y) Signifies sub-lethal epithelial effects in 100% (15 of 15 larvae) to zebrafish (Danio rerio) in 48 hour bioassay. (z) Signifies sublethal epithelial effects in ca. 50% of zebrafish (Danio rerio) (7 of 15 larvae) in 48 hour bioassay.

FIGS. 6A and B show (A) Reversed phase HPLC elution profile at 230 nm (dotted line) and 254 nm (solid line) for a 50 μl injection of a concentrated 70% MeOH extraction of filtered Karlodinium micrum (CCMP 1974) cells (400 ml; 5.5×10⁴ cells ml⁻¹). (B) Reversed phase HPLC elution profile at 230 nm (dotted line) and 254 nm (solid line) for a 50 μl injection of a concentrated 100% MeOH tC₁₈ elution of the culture filtrate from a Karlodinium micrum (CCMP 1974) culture (400 ml; 5.5×10⁴ cells ml⁻¹). For Both A and B the flow rate of separation was 1 ml min⁻¹. Overlaid histogram (gray bars) is the hemolytic activity, compared to cells lysed with 10 μg saponin, in 0.5 min. collected fractions.

FIGS. 7A and B show (A) Reversed phase HPLC elution profile at 230 nm (dotted line), and 254 nm (solid line) for a 50 μl injection of a concentrated 100% MeOH tC₁₈ elution of Karlodinium micrum (CCMP 1974) culture filtrate (400 ml; 5.5×10⁴ cells ml⁻¹). Flow rate of separation was 1 ml min⁻¹. (B) In vitro cytotoxicity assay, based on the release of lactate dehydrogenase, testing 0.5 min. reversed phase HPLC fractions of a concentrated 80% MeOH tC18 elution of Karlodinium micrum (CCMP 1974) culture filtrate (2 L; 3.0×10⁴ cells ml⁻¹). Cells tested were a GH(4)C(1) rat pituitary tumor cell line (ATCC CCL-82.2). LDH release is represented as the percentage of release compared to cells lysed with 10 μg saponin. Bars represent range (n=2).

FIG. 8 shows cell lysis in Karlodinium micrum (CCMP 1974) and Prorocentrum minimum (strain PM-1) cultures (3.54×10⁴ cells ml⁻¹) exposed to either CuSO₄ (0.5, 2, or 8 mg L⁻¹)[=0.2, 08, or 3.18 mg L⁻¹ Cu] or KMnO₄ (2, 4, or 16 mg L⁻¹). Mid-range values of CuSO₄ and KMnO₄ (2 L⁻¹and 4 mg L⁻¹, respectively) approximated dosages applied at HyRock fish farm, while upper-range values of CuSO₄ and KMnO₄ (8 mg L⁻¹ and 16 mg L⁻¹, respectively) were similar to published LC₅₀ values for aquaculture species under comparable water quality conditions. Cell lysis was determined by enumerating the 7-20 μm size fraction using a Coulter particle counter. Bars represent one standard error (n=3).

FIG. 9 shows the time course of hemolytic material release, measured as release of hemoglobin from rainbow trout erythrocytes, compared to cells lysed with 10 μg saponin, in Karlodinium micrum (CCMP 1974) cultures exposed to either CuSO₄ (2 mg L⁻¹)[=0.8 mg L⁻¹ Cu] or KMnO4 (4 mg L⁻¹). Cu exposures received EDTA (2 mM) prior to hemolysis testing to remove any hemolytic effects due to free Cu remaining in solution. Final cell concentrations in assay were equivalent to 1.5×10⁵ cells ml⁻¹. Bars represent one standard deviation (n=4).

FIG. 10 illustrates the hemolytic activity profile of karlotoxins KmTx 1, KmTx 2, KmTx 4, and KmTx 5.

FIG. 11 illustrates the elution profile of karlotoxin KmTx 1.

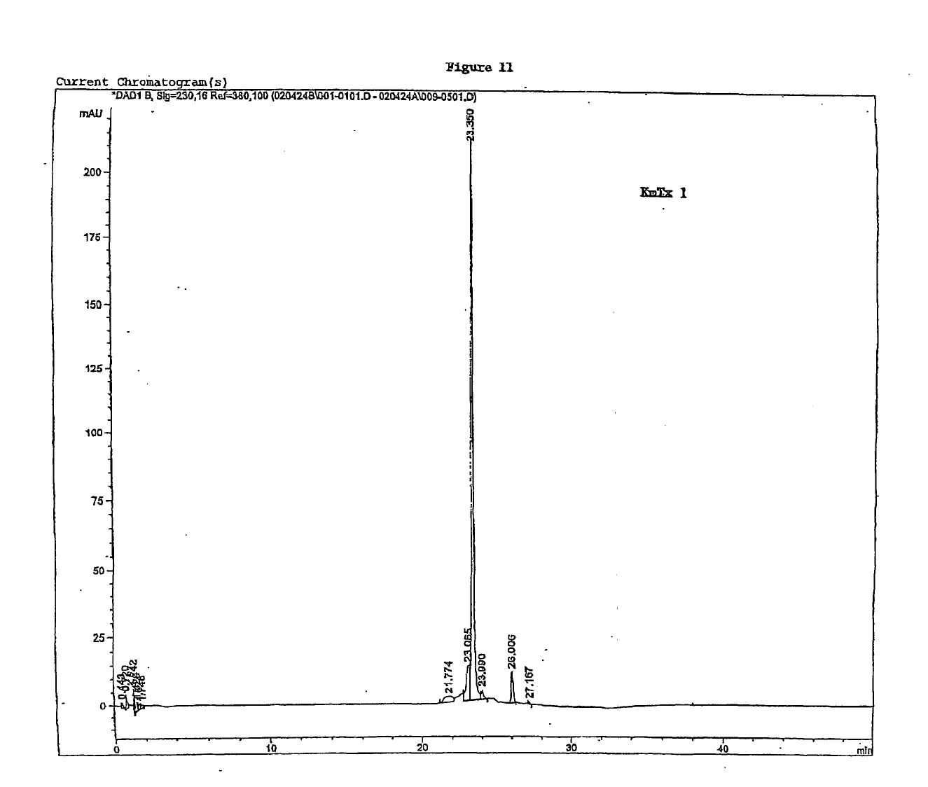

FIG. 12 illustrates the elution profile of karlotoxin KmTx 2.

FIG. 13 illustrates the elution profile of karlotoxin KmTx 3.

FIG. 14 illustrates the elution profile of karlotoxin KmTx 4.

FIG. 15 illustrates the elution profile of karlotoxin KmTx 5.

FIG. 16 illustrates the elution profile of karlotoxin KmTx 6.

FIG. 17 illustrates the mass spectra of KmTx 1.

FIG. 18 illustrates the mass spectra of KmTx 2.

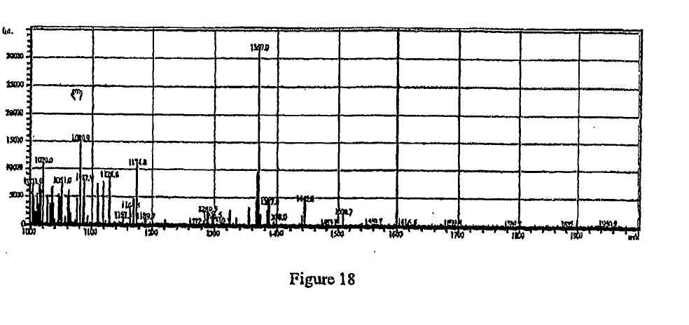

FIGS. 19A and B show (A) LC/MS trace of purified KmTx 2. Solid line [left axis]230 nm absorbance. Dashed line [right axis] mass intensity. (B) Negative ion mass spectra of KmTx 2.

FIGS. 20 A and B shows (A) HPLC trace of gymnodinosterol (ca. 80% pure) isolated from Karlodinium micrum. (B) GC/MS spectra of gymnodinosterol isolated from Karlodinium micrum.

FIG. 21 shows inhibition of hemolysis of rainbow trout erythrocytes due to exposure to 0.25, 0.5 or 1 μg/ml KmTx 2 after co-incubation with 30 mM of either sucrose (MW 342.3), polyethylene glycol (MW 400), polyethylene glycol (MW 600), maltohexaose (MW 990.0), polyethylene glycol (MW 8,000), dextran (MW 10,000).

FIGS. 22A, B and C show measurement of Ca²⁺ flux into rat embryonic fibroblast (REF 52) cells using the intracellular fluorescent indicator fura-2. (A) Addition of DMSO control followed by the addition of 0.25 4 μg/ml KmTx 2, note rapid increase in cytosolic Ca²⁺ levels followed by slight recovery. (B) Addition of 0.2 μM vasopressin followed by the addition of 1 μg/ml KmTx 2. For vasopressin, a carrier type Ca²⁺ ionophore, note spike in cytosolic Ca²⁺ levels followed by rapid decline to level above baseline. For KmTx 2 addition note rapid rise in cytosoloc Ca²⁺ levels with no recovery. (C) Comparison of time course of Ca²⁺ influx for 0.25 and 1 μg/ml KmTx 2 additions.

FIGS. 23 A, B, C and D show H&E stained sections of whole 60 day old zebrafish (Danio rerio) exposed to an increasing concentration of KmTx 2. (A) Control at 6 hrs. [a. eye, b. brain, c. skeletal muscle, d. pseudobranch, e. gills, f. thymus, g. skin] (B) Control gills at 6 hrs. (C) 0.1 μg/ml KmTx 2 exposure at 6 hrs. (D) 0.5 μg/ml KmTx 2 exposure at 1 hr. For B., C., and D. arrows indicate secondary gill lamellae. Bars equal 350 μm for A., and 50 μm for B., C., and D.

FIGS. 24A and B show (A) One hour exposure of Karlodinium micrum (CCW 2282) to an increasing concentration of KmTx 2. Histogram [left axis] represents number of K. micrum counted using a Coutler Multisizer II particle counter enumerating the 7-20 μm size fraction. Filled diamonds [right axis] represents hemolysis of rainbow trout erythrocytes due to KmTx 2 remaining in solution after one hour exposure. Lines represent one standard deviation (n=3) for both. (B) One hour exposure of a Chesapeake Bay isolate of Oxyhhris marina exposed to an increasing concentration of KmTx 2. Histogram [left axis] represents number of O. marina counted using a Coutler Multisizer II particle counter enumerating the 15-30 μm size fraction. Filled diamonds [right axis] represents hemolysis of rainbow trout erythrocytes due to KmTx 2 remaining in solution after one hour exposure. Lines represent one standard deviation (n=3) for both.

FIGS. 25A and B show hemolysis of rainbow trout erythrocytes due to exposure to 0, 0.1, 0.5, or 1 μg/ml KmTx 2 after co-incubation with (A) gymnodinosterol isolated from Karlodinium micrum, (B) ergosterol, and C. cholesterol. For all: [□]0, [◯]0.001, [Δ]0.01,[∇]0.1,[⋄]1,[D′]10 μM sterol.

### DETAILED DESCRIPTION OF PREFERRED EMBODIMENTS

In order to facilitate review of the various embodiments of the invention and provide an understanding of the various elements and constituents used in making and using the present invention, the following terms used in the invention description have the following meanings.

Definitions

The term “K. micrum toxin” or “Karlotoxin,” as used herein, is defined as any one of the six toxins produced by Karlodinium micrum and described herein and based upon their rates of elution from a C₁₈ High-Performance Liquid Chromatography (HPLC) column.

The term “isolated,” as used herein, is defined as separated from natural surroundings. The toxins of the instant disclosure can be found in the dinoflagellate as well as in water containing the dinoflagellate. An isolated dinoflagellate toxin would be one separated and purified from the dinofagellate and/or from the water where the dinoflagellate is found. Purification includes any increase in the percentage of purity greater than that found in a culture medium or a source of toxin. Sources of toxin include Karlodinium micrum or water containing Karlodinium micrum. Purity of an isolated toxin may be greater than 10%, and more preferably, above 50%, and most preferably, greater than 90% pure after purification from a source of toxin. Purification of Karlodinium micrum toxins is disclosed herein, using standard methods.

The term “medium,” as used herein is defined as any environment where dinoflagellates are growing. Medium also includes artificial culture mediums where dinoflagellates may be grown

As used herein, the term “antibody” refers to intact molecules as well as fragments thereof, such as Fa, F(ab′)₂, and Fv, which are capable of binding the karlotoxins.

The Invention

The present invention relates to isolating karlotoxins exhibiting toxic activity and characterizing such toxic activity associated with the dinoflagellate Karlodinium micrum.

In one embodiment the present invention provides antibodies reactive with the described karlotoxins of the present invention. Such antibodies include monoclonal, polyclonal, chimeric, and single chain antibodies. Antibodies further include all five antibody isotypes: IgG, IgM, IgA, IgD and IgE. Conjugation of antibodies is also well known in the art. The instant invention includes antibodies conjugated with the instant karlotoxins.

Means for preparing and characterizing antibodies are well known in the art. Briefly, a polyclonal antibody is prepared by immunizing an animal with an immunogen comprising a karlotoxin of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given toxin (polypeptide) may vary in its immunogenicity. It is often necessary therefore to couple the toxin of the present invention with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide or a polynucleotide to a carrier protein are well known in the art and include glutaraldehyde, M maleimidobenzoyl-N-hydroxysuccinimide ester, carbodiimide and bis-biazotized benzidine.

As is also well known in the art, immunogenicity to a particular immunogen karlotoxin of the present invention) can be enhanced by the use of non-specific stimulators of the immune response known as adjuvants. Exemplary and preferred adjuvants include complete Freund's adjuvant, incomplete Freund's adjuvants and aluminum hydroxide adjuvant.

The amount of immunogen used for the production of polyclonal antibodies varies inter alia, upon the nature of the immunogen as well as the animal used for immunization. A variety of routes can be used to administer the immunogen (subcutaneous, intramuscular, intradermal, intravenous and intraperitoneal. Sampling blood of the immunized animal at various points following immunization monitors the production of polyclonal antibodies. When a desired level of immunogenicity is obtained, the immunized animal can be bled and the serum isolated and stored. In another aspect, the present invention contemplates a process of producing an antibody reactive with a karlotoxin of the present invention comprising the steps of (a) transfecting recombinant host cells with polynucleotide that encodes for the karlotoxin peptide; (b) culturing the host cells under conditions sufficient for expression of the peptide; (c) recovering the peptide; and (d) preparing the antibodies to the peptide.

Typically, a monoclonal antibody of the present invention can be readily prepared by a technique which involves first immunizing a suitable animal with a selected karlotoxin antigen in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell. The fused spleen/myeloma cells are cultured in a selective medium to select fused spleen/myeloma cells from the parental cells. Fused cells are separated from the mixture of non-fused parental cells, for example, by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine. This culturing provides a population of hybridomas from which specific hybridomas are selected. Typically, selection of hybridomas is performed by culturing the cells by single-clone dilution in microliter plates, followed by testing the individual clonal supernatants for reactivity with an antigen-polypeptides. The selected clones can then be propagated indefinitely to provide the monoclonal antibody.

By way of specific example, to produce an antibody of the present invention, mice are injected intraperitoneally with between about 1-200 ug of an antigen, such as a karlotoxin of the present invention. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant. A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the karlotoxins of the present invention. Fusion conditions include, for example, the presence of polyethylene glycol. The resulting fused cells are hybridoma cells. Like myeloma cells, hybridoma cells grow indefinitely in culture. Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media. Each of the surviving hybridoma cells produces a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific karlotoxins of the invention can be recognized as antigens, and thus identified. Once identified, those karlotoxins can be isolated and purified by techniques such as antibody-affinity chromatography. In antibody-affinity chromatography, a monoclonal antibody is bound to a solid substrate and exposed to a solution containing the suspect karlotoxin. A karlotoxin is removed from the culture solution through an specific reaction with the bound antibody. The bound karlotoxin is then easily removed from the substrate and purified.

In another embodiment, the present invention provides pharmaceutical compositions exhibiting cytotoxic activity for killing and/or reducing the growth of cancer cells, the composition comprising a karlotoxin of the present invention and a physiologically acceptable carrier.

Compositions include compositions useful in pharmaceutical applications, and those useful as reagents, diagnostics and biological standards. Pharmaceutical compositions include karlotoxins individually or as a mixture, antibodies against said karlotoxins, mixtures of said antibodies, immunoconjugates comprising said Karlotoxins, and mixtures of said immunoconjugates with pharmaceutically acceptable carriers. Pharmaceutically acceptable carriers include those approved for use in animals and humans and include diluents, adjuvants, excipients or any vehicle with which a compound is administered.

A composition of the present invention may be administered parenterally in dosage unit formulations containing standard, well-known nontoxic physiologically acceptable carriers, adjuvants, and vehicles as desired. The term parenteral as used herein includes intravenous, intramuscular, intraarterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables. Preferred carriers include neutral saline solutions buffered with phosphate, lactate, Tris, and the like.

The compositions may further comprise adjuvants including but are not limited to alum, mineral oil, cholera toxin b-subunit, dehydroepiandrosterone sulfate, Freund's (complete and incomplete), lysolecithin, pluronic polyols, keyhole limpet hemocyanin, dinitrophenol, Bacillus Calmette-Guerin and Corynebacterium parvum.

Pharmaceutical compositions may also include wetting or emulsifying agents, or pH buffering compounds. Wetting or emulsifying agents include, but are not limited to, sodium dodecyl sulfate, polyoxyethylene derivatives of fatty acids, partial esters of sorbitol anhydrides, TWEEN 80, TWEEN 20, POLYSORBATE 80, TRITON X 100, bile salts such as sodium deoxycholate, zwitterionic detergents such as N-dodecyl-N, N-dimethyl-2-ammonio-1 ethane sulphonate and its congeners or non-ionic detergents such as octyl-beta-D-glucopyranoside.

Pharmaceutically acceptable buffers are known in the art and include but are not limited to sodium phosphate, sodium citrate, sodium acetate, TRIS glycine, HEPES, MOPS or Bis-Tris

In yet another aspect, the present invention contemplates a process of screening substances for their ability to interact with a karlotoxin of the present invention, the process comprising the steps of providing a karlotoxin of the present invention and testing the ability of selected substances to interact with that karlotoxin.

Screening assays of the present invention generally involve determining the ability of a candidate substance to bind to or modulate the activity of the karlotoxin of the present invention, The karlotoxins of the present invention can be coupled to a solid support. The solid support can be agarose beads, polyacrylamide beads, polyacrylic beads or other solid matrices capable of being coupled to karlotoxin proteins of the present invention. Well known coupling agents include cyanogen bromide, carbonyidiimidazole, tosyl chloride, and glutaraldebyde.

Accordingly, it is proposed that this aspect of the present invention provides those of skill in the art with methodology that allows for the identification of karlotoxins in an admixture suspected of including karlotoxins. An antibody specific for the karlotoxins of the present invention and a culture medium comprising a suspect karlotoxin is allowed to incubate for a selected amount of time, and the resultant incubated mixture subjected to a separation means to separate the unbound compounds remaining in the admixture from any karlotoxin/antibody complex so produced. Then, one simply measures the amount of each (e.g., versus a control to which no candidate substance has been added). This measurement can be made at various time points where velocity data is desired.

Numerous techniques are known for separating the unbound compounds from the karlotoxin/antibody complex, and all such methods are intended to fall within the scope of the invention. Use of thin layer chromatographic methods (TLC), HPLC, spectrophotometric, gas chromatographic/mass spectrophotometric or NMR analyses. It is contemplated that any such technique can be employed so long as it is capable of differentiating between the unbound compounds and complex.

The present invention provides a process of screening a biological sample for the presence of a karlotoxin. A biological sample to be screened can be a biological fluid such as extracellular or intracellular fluid or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide.

In accordance with a screening assay process, a biological sample is exposed to an antibody reactive with the karlotoxins whose presence is being assayed. Typically, exposure is accomplished by forming an admixture in a liquid medium that contains both the antibody and the candidate karlotoxin. Either the antibody or the sample with the karlotoxin can be affixed to a solid support (e.g., a column or a microliter plate). The biological sample is exposed to the antibody under biological reaction conditions and for a period of time sufficient for antibody-karlotoxin conjugate formation. Biological reaction conditions include ionic composition and concentration, temperature, pH and the like. Ionic composition and concentration can range from that of distilled water to a 2 molal solution of NaCl. Temperature preferably is from about 25 ° C. to about 40 ° C. pH is preferably from about a value of 4.0 to a value of about 9.0, more preferably from about a value of 6.5 to a value of about 8.5 and, even more preferably from about a value of 7.0 to a value of about 7.5. The only limit on biological reaction conditions is that the conditions selected allow for antibody-karlotoxin conjugate formation and that the conditions do not adversely affect either the antibody or the peptide.

Exposure time will vary inter alia with the biological conditions used, the concentration of antibody and karlotoxin (peptide) and the nature of the sample (e.g., fluid or tissue sample). Means for determining exposure time are well known to one of ordinary skill in the art. Typically, where the sample is fluid and the concentration of peptide in that sample is about 10⁻¹⁰ M, exposure time is from about 10 minutes to about 200 minutes.

The presence of a karlotoxin in the sample is detected by detecting the formation, and presence of antibody-peptide conjugates. Means for detecting such antibody-antigen (e.g., karlotoxin) conjugates or complexes are well known in the art and include such procedures as centrifugation, affinity chromatography and the like, binding of a secondary antibody to the antibody-karlotoxin candidate peptide complex.

In one embodiment, detection is accomplished by detecting an indicator affixed to the antibody. Exemplary and well known such indicators include radioactive labels (e.g., ³²P, ¹²⁵I, ¹⁴C), a second antibody or an enzyme such as horse radish peroxidase. Means for affixing indicators to antibodies are well known in the art. Commercial kits are available.

The following examples are intended to illustrate but not limit the present invention.

### EXAMPLE 1

On Feb. 5, 2002, a tidally influenced 7-acre retention pond associated with a housing complex in Mount Pleasant, S.C. (near Charleston) was sampled in response to a reported fish kill. Dying fish were observed by a citizen on the evening of Feb. 3, 2002 and the kill event continued through at least Feb. 5, 2002, at which time it was reported to the South Carolina Harmful Algal Bloom Program (SCHABP). Light and epifluorescence microscopic analyses on collected water revealed the presence of high concentrations (68,280 cell ml⁻¹) of a dinoflagellate that was tentatively identified as K. micrum. A combination of morphological, biochemical, and molecular diagnostics were used to assess species identity of the bloom dinoflagellate, and measure toxicity of filtered water. The results provide compelling evidence for toxic K. micrum as a causative factor in a South Carolina brackish retention pond fish kill.

Material and Methods

Toxicity assays

The filtrate from water samples collected on Feb. 5 and 6, 2002 from the Mount Pleasant pond fish kill event, and from the K. micrum culture (strain 010410-C6) isolated from a Hilton Head pond were tested for hemolytic and ichthyotoxic activity. Environmental water samples were allowed to thaw to room temperature and passed through a Sep-Pak Plus tC₁₈ disposable cartridge (Waters Corp., Milford Mass.), attached to a vacuum manifold. The column was pre-equilibrated with methanol (20 ml) followed by H₂O (20 ml). The cartridges were eluted with the following concentrations of MeOH/H₂O: 100% H₂O, 40%, 60%, 80%, and 100% MeOH (12 ml ea⁻¹). An aliquot of each elution was diluted, using Tris buffer +CaCl₂, to give final dilutions ranging from 6 to 60% of original concentrations. These samples were tested for hemolytic activity based on the lysis of rainbow trout (Oncorhynchus mykis) erythrocytes (following Deeds et al. 2002). The occurrence of hemolytic activity was determined by the absorbance of released hemoglobin, measured at 540 nm. Saponin (10 μg) served as a positive hemolysin control. Treatments were run in quadruplicate.

Based on the results of the preceding hemolytic analyses, the 80% MeOH C₁₈ column elution was concentrated using a SpeedVac (Savant AES1010), then freeze-dried overnight. The dehydrated material was resuspended in MeOH (1 ml), re-dryed under N₂ gas, weighed, then resuspended once again in MeOH (1 ml). An aliquot (25 μl) of each suspension was injected onto a LiChroDART 125-4/RP-8 (5 μm) reversed phase HPLC column (Waters Corp., Milford Mass.) and eluted with a 95% H₂O /5% MeOH to 5% H₂O /95% MeOH linear gradient, over 20 min., at a flow rate of 1 ml min⁻¹ (Hewlett Packard Series 1100 HPLC System, Hewlett Packard Corp., Wilmington Del.). Fractions were collected every 0.5 min and assayed for hemolytic activity.

Based on hemolytic screening and spectral analysis, two HPLC fractions, fractions 45 and 46 corresponding to an elution time of ca. 22 min, were combined, evaporated to dryness under N₂ gas, resuspended in MeOH (50 μl), and tested for ichthyotoxic activity at concentrations equivalent to 10% of original (following Deeds et al. 2002). Ichthyotoxicity was determined using a bioassay measuring percent mortality of 48-hr old post-hatch zebrafish (Danio rerio) larvae after exposure to the eluted fractions indicated above.

Results

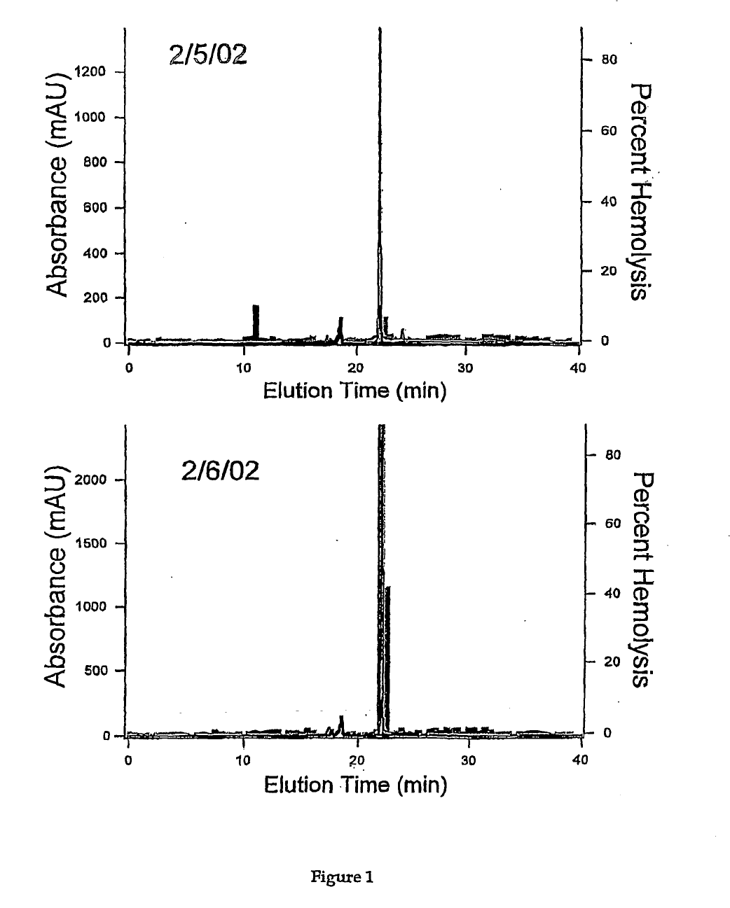

High hemolytic activity was detected both in the filtered water samples from Feb. 5 and 6, 2002, and in the 80% MeOH elution from the C₁₈ columns. The dehydrated 80% MeOH C,₈ elutions from Feb. 5 and 6, 2002 (original samples were 650 and 1000 ml, respectively) yielded 0.98 and 1.63 mg of organic material. Reversed phase HPLC analysis of an aliquot of these concentrated 80% MeOH C₁₈ elutions revealed a prominent peak eluting ca. 22 min in both samples. These peaks were further shown to be associated with high hemolytic activity (FIG. 1).

Zebrafish larvae exposed to this same 22 min peak died in <5 min for both samples (15 of 15 larvae). Unexposed controls and controls exposed to an equivalent amount of MeOH showed no adverse effects after 24 hours.

Using the filtrate from water collected during a fish kill coincident with a bloom (>6×10⁴ cells ml⁻¹) of Karlodinium micrum, high hemolytic activity was demonstrated, apparently due to a polar lipid-like compound eluting ca. 22 min upon reversed phase HPLC separation. This fraction was further shown to be ichthyotoxic to zebrafish larvae even at equivalent concentrations well below ambient (i.e. after dilution). These results provide good evidence for K. micrum toxicity as a causative factor in the Mount Pleasant, S.C. subdivision pond fish kill.

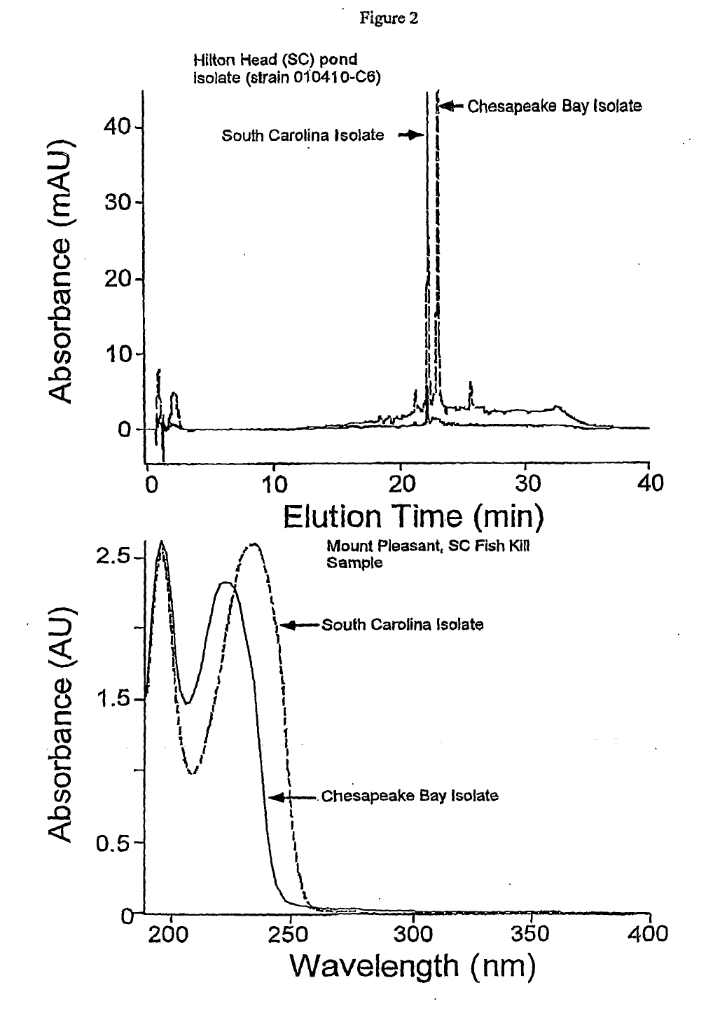

Based on retention time and UV spectral analysis, the compound isolated from both the Mount Pleasant, S.C. water samples and the Hilton Head Island, S.C. culture isolate (strain 010410-C6) were not identical to the ones isolated from the Chesapeake Bay cultures. To confirm this, aliquots of hemolytic HPLC fractions from South Carolina isolate 010410-C6 and Chesapeake Bay isolate CCMP 1974 were co-injected, using the above described reversed phase HPLC procedure, and they still eluted as two distinct peaks (FIG. 2).

### EXAMPLE 2

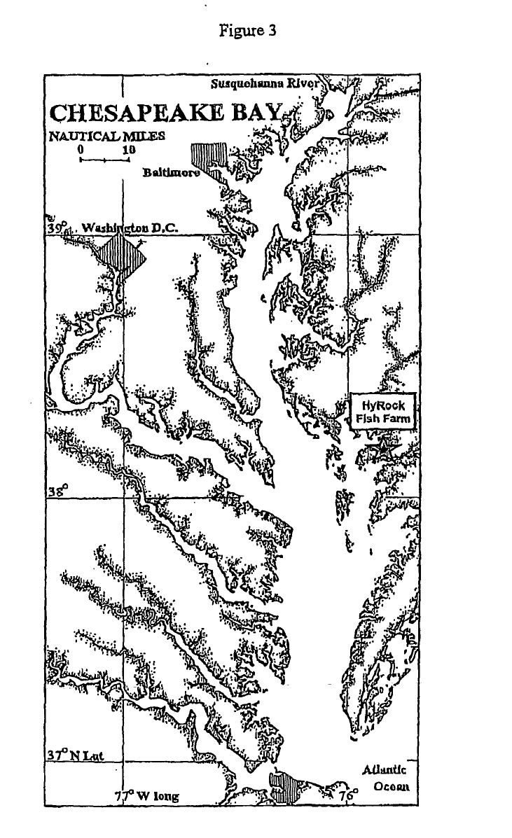

HyRock Fish Farm was opened in 1993 and consists of 37 acres of impoundments supplied with water from the Manokin River, a tributary of the Chesapeake Bay located in Princess Anne, Md., USA FIG. 3). Average salinity of the incoming Manokin river water is 10 psu (range 4.5-18 psu).

On Jul. 30, 1996 a large mortality of ca. 15,000, 1-1.25 lb. (2.20-2.75 kg) reciprocal cross hybrid striped bass (Morone saxatilis male×Morone chrysops female) occurred following a copper sulfate treatment (<2 mg L⁻¹) to arrest a dense dinoflagellate bloom. The bloom had been developing for over one week prior to the events of Jul. 30, 1996 but no mitigating actions were taken due to the misdiagnosis of the deep mahogany water coloration as tannins. Limited fish mortalities had occurred in the days preceding Jul. 30, 1996. To avoid low dissolved oxygen problems, paddle wheel aeration was initiated maintaining dissolved oxygen levels >5 ppm. Alkalinity was 75 ppm and other water quality conditions immediately prior to the main kill were within normal mid-summer ranges for HyRock Fish Farm (see Glibert and Terlizzi, 1999). Foaming and a “petroleum-like” odor were present in the days preceding the large kill. Treatment using potassium permanganate (<4 mg L⁻¹), a strong oxidizing agent, of a neighboring pond with similar mixed dinoflagellate populations, appeared to arrest the bloom without fish mortality.

The bloom was subsequently determined to be dominated by the 10-15 μlm, non-thecate, mixotrophic dinoflagellate Karlodinium micrum, originally identified as Gyrodinium estuariale, (ca. 6×10⁴ cells ml⁻¹), with <1,000 cells ml⁻¹ of an unidentified dinoflagellate (Gymnodinium sp.) and several additional <10 μm unidentified species (Wayne Coats, Smithsonian Environmental Research Center, Edgewater Md., personal communication). Some of the <10 μm dinoflagellates present resembled life history stages of Pfiesteria piscicida, as described in Burkholder et al. (1992) and Steidinger et al. (1996), so samples were forwarded to the laboratory of Dr. Karen Steidinger (Florida Marine Research Institute, Florida Fish and Wildlife Conservation Commission, St. Petersburg Fla.) who confirmed through SEM and light microscopy that P. piscicida was present in the sample (ca. 300 cells ml⁻¹). Pathological examination of both live and preserved specimens at the time of the kill suggested that suffocation due to gill inflammation was the cause of mortality, although brain tissue also showed some abnormalities (Eric May, University of Maryland Eastern Shore, Salisbury Md., personal communication). Since that time, two additional fish mortality events have co-occurred with blooms of this organism; 8000 adults on Aug. 13, 1997, and 5000 fingerlings on May 15, 1999. In both circumstances, mortality was stopped following a <4 mg L⁻¹ potassium permanganate treatment, with fish returning to feed in 3-5 days.

To determine that Karlodinium micrum was in part responsible for the mortalities of hybrid striped bass that co-occurred with blooms of this organism at HyRock Fish Farm since 1996, we tested isolates from the Hyrock Fish Farm.

Materials and Methods

The following dinoflagellate isolates were chosen for initial toxicity screening: Karlodinium micrum (Leadbeater and Dodge) J. Larsen (CCMP 1974; Chesapeake Bay isolate) and (CCMP 1975; HyRock Fish Farm isolate). For comparison purposes, a Maryland isolate of Prorocentrum minimum (strain PM-1; provided by the University of Maryland Center for Environmental and Estuarine Science Horn Point Laboratory, Cambridge Md.), a North Carolina P. minimum isolate (provided by Patricia A. Tester, NOAA Center for Coastal Fisheries and Habitat Research, Beaufort N.C.), Cryptoperidiniopsis sp. (CCMP 1828; Chesapeake Bay isolate), and Pfiesteria piscicida (CCMP 1921; Chesapeake Bay isolate), were also screened for hemolytic activity. The P. piscicida isolate used in this study, original designation M CC #981020BR01C5, was a gift from Karen Steidinger, Florida Marine Research Institute, and has been maintained on algae since its arrival on Dec. 17, 1998. Additional species tested were the cryptophytes Rhodomonas sp. (CCMP 767) and Storeatula major (strain g; Chesapeake Bay isolate) (used in lipid class separation experiments only), two commonly used food sources for heterotrophic and mixotrophic dinoflagellates in this size class (10-20 μm).

Culturing

Karlodinium micrum (CCMP 1974 and 1975) and Prorocentrum minimum (strain PM-1 and the North Carolina isolate) all clonal but not axenic, were cultured autotrophically in 12 psu, filtered (0.22 μm), artificial sea water (ASW) (Instant Ocean Brand), with added f/2-Si nutrient mixture (Guillard 1975), and 1.5% soil extract and 0.3% chicken manure extract. Soil and chicken manure extracts were produced by autoclaving (121° C. and 15 psi) either 400 g of air dried soil, not previously exposed to pesticides or fertilizers, or 50 g of air dried chicken manure, in 1L of distilled-deionized (ddi) water for 1 hour. Mixtures were allowed to sediment for 24 hours, decanted, and the supernatant was further centrifuged to remove remaining particulates. The pH of the extract was adjusted to 6.8-7.0, then filter sterilized (0.22 μm) and stored at 4° C. The additional extracts were added to simulate the nutrient rich, i.e. high DON, environment typical of fish farms. Cultures were maintained at 20° C., with an alternating 12 hour light/dark cycle with 100-120 μmol m⁻² s⁻² of illumination, measured using a Li-COR model LI-250 light meter with a LI-190S Quantum sensor (Li-COR, inc., Lincoln Nebr.).

Pfiesteria piscicida (CCMP 1921) and Cryptoperidiniopsis sp. (CCMP 1828) were grown in 15 psu ASW (Instant Ocean Brand), with added f/2-Si nutrient mixture (Guillard 1975), at 20° C., and 170 μmol m⁻² s⁻¹ illumination, with an alternating 12 hour light/ 12 hour dark cycle, using Rhodomonas sp. (CCMP 767) as a food source. P. piscicida (CCMP 1921) and Cryptoperidiniopsis sp. (CCMP 1828) were starved for 48 hours prior to all experiments to reduce the number of food organisms. Rhodomonas sp. (CCMP 767) was grown at 32 psu under the same conditions as described for P. piscicida and Cryptoperidinopsis sp.

Hemolytic and ichthyotoxic activity were assayed in both lysed and non-lysed cultures of Karlodinium micrum (CCMP 1974) and Prorocentrum minimum (strain PM-1). Hemolytic activity alone was assayed in lysed and non-lysed cultures of K. micrum (CCMP 1975), Pfiesteria piscicida (CCMP 1921), Cryptoperidiniopsis sp. (CCMP 1828), Rhodomonas sp. (CCMP 767), and P. minimum (North Carolina isolate). All cultures were between 1.5-2.5×10⁵ cells ml⁻¹, with the exception of K. micrum (CCM 1975) (5×10⁴ cells ml⁻¹) which was assayed immediately upon arrival to test for culturing artifacts. Rhodomonas sp. (CCMP 767) cultures were diluted to 15 psu with ddi H₂O. Cultures were lysed through a pulsed sonication (30 sec. on/30 sec. off), on ice, for 5 minutes using a microtip sonicator (50 Watt, 3 mm tip, 60 amplitude). Cultures were confirmed to be >70% lysed by using a Coulter Multisizer II particle counter with enumeration of the 7-20 μm size fraction using a Coulter Accucomp software package (Coulter Electronics Limited, Miami Fla.).

Hemolytic Assay

A hemolytic assay based on the lysis of fish erythrocytes was utilized to screen for bioactive materials. Cultures and culture fractions that were positive in the hemolytic assay were tested further using assays for ichthyotoxicity and cytotoxicity.

Erythrocyte suspensions were prepared as described in Edvardsen et al. (1990). Blood was extracted from the caudal vein of rainbow trout (Oncorhynchus mykis) provided by the Center of Marine Biotechnology's Aquaculture Research Center. Needles were heparin (Sigma Chemical Co., St. Louis Mo.) treated and 10 units ml⁻¹ of additional heparin was added to whole blood samples to prevent clotting. Erythrocyte suspensions were prepared by washing three times (2500 g for 5 min.) with ice cold buffer [150 mM NaCl, 3.2 mM KCl, 1.25 mM MgSO₄, and 12.2 mM Tris base]. Buffer pH was adjusted to 7.4 at 10° C. with 1N HCl, then filter sterilized (0.22 μm). After the third wash, cells were stored in the Tris buffer with 3.75 mM CaCl₂ at 50% of their original concentration. Suspensions were stored at 4° C. for no longer than 10 days.

Hemolytic assays were performed by diluting test material in Tris buffer +CaCl₂ (100 μl total) and adding this to a 5% erythrocyte suspension (100 μl). Assays were run in 96 well, V-bottom, non-treated, polystyrene plates (Corning Inc., Corning N.Y.) sealed with Falcon 3073 pressure sensitive film (Becton Dickinson Labware, Lincoln Park N.J.). Assays were incubated on an orbital shaker (80-100 rpm) at 20° C. for 1 hour. Plates were then centrifuged at 2500 g for 5 min. and the supernatant (100 μl) was transferred to another flat bottom 96 well polystyrene plate, where the absorbance of released hemoglobin was read at 540 nm. Saponin (10 μg) (from Quillaja bark; Sigma Chemical Co., St. Louis Mo.) was used as a positive hemolysin control All treatments were run in quadruplicate.

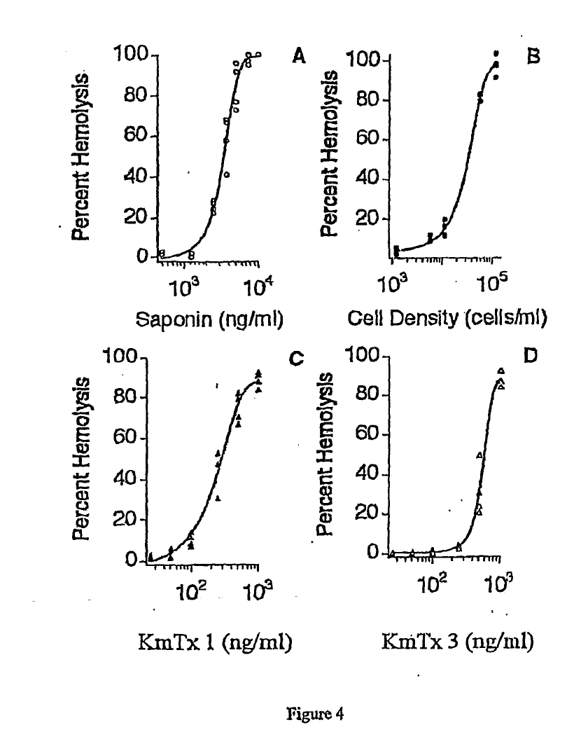

Throughout the course of these experiments, hemolytic activity was consistently observed in sonicated cultures of Karlodinium micrum (CCMP 1974 and 1975) (0.1-2.5×10⁵ cells ml⁻¹). Filtering small volumes (>5 ml) of either whole or sonicated Karlodinium micrum suspensions through filters composed of nylon (0.22 μm), hydrophobic PTFE (0.22 μm), or glass fibers (0.7 μm) was found to remove or greatly reduce hemolytic activity. Hemolytic activity was still present in supernatants after a 20 min. centrifugation at 16,000 g at 4° C. Filtering either larger volumes of culture through glass fiber filters (0.7 μm) or any volume of the aq-MeOH C₁₈ elutions through either nylon or hydrophobic PTFE membranes (0.22 μm) had little or no affect on hemolytic activity. No hemolytic activity was observed in either whole or sonicated cultures of Prorocentrum minimum (Maryland and North Carolina isolates), Cryptoperidiniopsis sp. (CCMP 1828), Pfiesteria piscicida (CCMP 1921), or Rhodomonas sp. (CCMP 767), all 1.5×10⁵ cells ml⁻¹. Hemolytic activity in whole K. micrum cultures (non-sonicated) was highly variable, ranging from 0% to >80% lysis of rainbow trout erythrocytes, and did not appear to be correlated exclusively with K. micrum cell number (data not shown). Hemolytic activity in sonicated cultures was detectable (>10%) in dilutions equivalent to ca. 5000 cells ml⁻¹ (FIGS. 4 A-D).

Ichthyotoxicity Assay

Ichthyotoxicity was evaluated using a static, acute (24-48 hour), small volume (2 ml), larval fish bioassay. Exposures were performed at 20° C. in 24- well non-tissue culture treated polystyrene plates (Becton Dickinson Labware, Franklin Lakes N.J.). Two species were utilized for ichthyotoxicity testing, zebrafish (Danio rerio) and sheepshead minnows (Cyprinodon variegatus), depending on their availability and salinity tolerance, with sheepshead minnows being tolerant to a wider range of salinities, but zebrafish being more readily obtainable in large numbers. Therefore, zebrafish, 5-<48 hrs old post-hatch, were used for toxic fraction testing, while sheepshead minnows, 3-<24 hours old post-hatch, were used to test whole dinoflagellate cultures. Larvae were not fed prior to or during testing. Previous experiments had shown that, at the biomass/water ratios used for each species, oxygen saturation remained >60% during the 48 hour exposure, as recommended in the Standard Guide for Conducting Acute Toxicity Tests with Fishes, Macroinvertebrates, and Amphibians (ASTM, 1992). All treatments were run in triplicate.

Ichthyotoxicity (100%, 9 of 9 larvae) was observed in sonicated but not whole cultures of K. micrum (CCMP 1974) (1.5×10⁵ cells ml⁻¹) using the static, acute, 24 hour bioassay with sheepshead minnow (Cyprinodon variegatus) larvae. At 5 min. post exposure, effects at the pectoral and caudal fins in sonicated K. micrum exposures were noticeable. At 1 hour post-exposure, severe epithelial damage/sloughing was observed, including complete destruction of the pectoral and caudal fins, but heartbeat and peripheral circulation were still present.

Cytotoxicity Assay

Cytotoxicity was assessed using an in-vitro toxicology assay kit based on the release of lactate dehydrogenase (LDH) (TOX-7, Sigma Chemical Co., St. Louis, Mo.). A GH(4)C(1) rat pituitary tumor cell line (ATCC, CCL-82.2) was utilized for the assay. The GH(4)C(l) cell line has previously been shown to be sensitive to several marine algal toxins (Young et al., 1995; Xi et al., 1996; Fairey et al., 1999). Saponin (10 μg) was used as a positive control. The assay was run in duplicate, and according to the manufacturers instructions.

The LC₅₀ for hemolysis of a sonicated cell suspension was 2.4×10⁴ cells ml⁻¹, well within the range of cell concentrations observed associated with fish kills. The toxic activity from K. micrum cells and culture filtrates was traced to two distinct fractions that co-elute with polar lipids. The LC₅₀ for hemolysis of the larger of these two fractions (KmTx 1) was 284 ng ml⁻¹ while the LC₅₀ of the second, smaller, fraction (KmTx 3) was 600 ng ml⁻¹. For comparison, the LC₅₀ for the standard hemolysin saponin was 3203 ng ml⁻¹. At concentrations of 800 and 2000 ng ml⁻¹, respectively, KmTx 1 was further shown to be ichthyotoxic to zebrafish (Danio rerio) larvae (80% mortality), and cytotoxic to a mammalian GH(4)C(1) cell line (100% LDH release). At a concentration of 600 ng ml⁻¹ KmTx 3 was shown to be cytotoxic to a mammalian GH(4)C(1) cell line (>30% LDH release), but not ichthyotoxic to zebrafish (Danio rerio) larvae up to a concentration of 250 ng ml⁻¹.

Isolation of Toxic Fractions from Karlodinium micrum Cultures

Lipid Class Separation

Total lipids and separated lipid classes were obtained following procedures described in Parrish et al. (1998) and Yongmanitchi and Ward (1992). All solvents used were HPLC grade or equivalent. To obtain the total lipid fraction, cultures of K. micrum (CCMP 1974) and P. minimum (North Carolina isolate) (ca. 1×10⁸ total cells ea⁻¹) were filtered onto pre-combusted type GF/F filters (Whatman International Ltd., Maidstone England) and total lipids were extracted through washes (3×4 ml ea⁻¹) with 2:1, 1:1, and 1:2 CH₂Cl₂/ MeOH. Each wash consisted of a 30-minute incubation in a Branson 1200 sonicated water bath (Branson Ultrasonics Corp., Danbury Conn.). Next, to the combined washes (ca. 12 ml total) was added 25% of the volume (ca. 3 ml) of a 0.88% KCl solution and vortexed to mix. The mixture was centrifuged at low speed to separate, and the upper phase was removed and discarded. The lower phase was dried under nitrogen, resuspended in CHCl₃ (1 ml), and stored at −20 ° C. until further separation.

Lipid class separations were performed using disposable silica cartridges (Sep-Pak Plus Silica, Waters Corp., Milford Mass.). The silica cartridge was attached to a vacuum manifold and equilibrated with MeOH (20 ml) followed by CH₃Cl₂ (2×15 ml). The lipid extract (c 3 mg lipid), in CHCl₃, was then loaded onto the column. The elution procedure and expected products, based on the results of Yongmanitchi and Ward (1992) for Phaeodactylu tricornutum, were as follows: (i.) 7.5 ml CHCl₃ eluting neutral lipids, pigments, chlorophylls, and carotenoids; (ii.) 9 ml CHCI₃/acetone (11:9) eluting monogalactosly-diacylglycerol (MGDG); (iii.) 9 ml CHCl₃/acetone (7:13) eluting digalactosyl-diacylglycerol (DGDG); (iv.) 15 ml CHCl₃/MeOH (7:1) eluting sulfoquinovo-diacylglycerol; (v.) 9 ml CHCI₃ / MeOH (7:3) eluting an unknown acyl-lipid; (vi.) 9 ml CHCI₃/MeOH (1:1) eluting phosphatidylcholine (PC), and finally (vii.) 6 ml MeOH eluting lysophospholipid (LC). After elution, each fraction was evaporated to dryness in a Savant SpeedVac AES1010 concentrator (Savant Instrument Inc., Farmingdale N.Y.) at 40° C., resuspended in MeOH (1 ml), and stored at −80° C. until tested for toxicity. Hemolytic and ichthyotoxic activity was assayed for in aliquots of the seven lipid classes obtained from K. micrum (CCMP 1974) (=4.78×10⁷ cells ml⁻¹) and P. minimum (North Carolina isolate) (=6.70×10⁷ cells ml⁻¹). Zebrafish were used for ichthyotoxicity testing of lipid fractions, as previously described, using aerated, reconstituted fresh water (soft) pH 7.3-7.5, hardness 40-48 mg L⁻¹ CaCO₃, alkalinity 30-35 mg L⁻¹ CaCO₃ as the diluent (ASTM, 1992). In some cases, lipid samples were concentrated to reduce MeOH additions in the assay to <1%. In a separate experiment, hemolytic and ichthyotoxic activity was assayed for in aliquots of lipid classes, separated using the above mentioned procedure, from K. micrum (CCM 1974), grown both autotrophically (=1.55×10⁷ cells ml⁻¹) and mixotrophically (=1.0×10⁷ cells ml¹), using the cryptophyte Storeatula major (strain g) as the food source. Hemolytic and ichthyotoxic activity was also assayed for in aliquots of separated lipid classes from S. major (=3.44×10⁷ cells m⁻¹).

Isolations from Cells and Culture Filtrates

An exponentially growing culture of K. micrum (CCMP 1974) (400 ml; 5.5×10⁴ cells ml⁻¹) was filtered (at 15 in. Hg) onto a pre-combusted type GF/F filter (Whatman International Ltd., Maidstone England), and cells were extracted through a 30 second sonication on ice using a microtip sonicator (50 Watt, 3 mm tip, amplitude 60) in 70% MeOH (3×4 ml). The culture filtrate was saved and stored at −80° C. for later analysis. The supernatants from the three sonication steps were combined (12 ml total) and placed in a glass separatory funnel. The sonicated extract was then washed with both hexane (C₆H₁₄) and methylene chloride (CH₂Cl₂) (3×12 ml ea⁻¹). Hexane partitioned to the top phase, while methylene chloride partitioned to the bottom. Appropriate washes were combined, evaporated to dryness at 50° C. in a rotavapor (Buchi model R110, Switzerland), and resuspended in methanol (12 ml). The hemolytic activity was measured in the original aq-methanol extract, the hexane extract, the methylene chloride extract, and in the aq-MeOH fraction remaining after the hexane and methylene chloride washes.

The saved culture filtrate (thawed and at room temp.) was passed through a Sep-Pak Plus tC₁₈ disposable cartridge (Waters Corp., Milford Mass.), attached to a vacuum manifold. The column was pre-equilibrated with methanol (20 ml) followed H₂O (20 ml). The cartridge was subsequently eluted with increasing concentrations of MeOH /H₂O as follows: 100% H₂O, 5%, 10%, 20%, 40%, and 100% MeOH (12 ml ea⁻¹). In a second experiment, another K. micrum (CCMP 1974) culture (2 L; 3.0×10⁴ cells ml⁻¹) was processed, as previously described, and eluted with 40%, 60%, 80%, and 100% MeOH solutions (15 ml ea⁻¹). An aliquot of each elution from the two experiments was diluted back to its original culture concentration (=5.5×10⁴ or 3.0×10⁴ cells ml⁻¹ for the first and second experiments, respectively) and tested for hemolytic activity as previously described.

The hemolytic extracts isolated from both the K. micrum cells and from the culture filtrate were dried under N₂ gas, weighed, and the material resuspended in MeOH (500 μl). An aliquot (50 μl) of each suspension was injected onto a LiChroDART 125-4/RP-8 (5 μm) reversed phase HPLC column (Waters Corp., Milford Mass.) and eluted with a 95% H₂O/5% MeOH to 5% H₂O /95% MeOH linear gradient, over 20 min., at a flow rate of 1 ml min⁻¹ (Hewlett Packard Series 1100 HPLC System, Hewlett Packard Corp., Wilmington Del.). Fractions were collected every 0.5 min. and assayed for hemolytic activity. Cytotoxic activity was evaluated, as previously described, for all 80 fractions at a cellular equivalent of 6.0×10⁵ cells ml⁻¹. As a negative control, MeOH (50 μl) was injected onto the column and fractions were collected and tested for both hemolytic and cytotoxic activity.

Based on hemolytic and cytotoxic screening, three HPLC fractions, fractions 36, 46, and 47, corresponding to elution times of 18, 23, and 23.5 min, respectively, were further tested for ichthyotoxicity. Ichthyotoxic testing was performed using zebrafish larvae, as previously described, at a cell equivalent of 2.4×10⁵ K. micrum cells ml⁻¹.

Based on spectral analyses, fractions 46 and 47 were combined, and along with fraction 36, were evaporated to dryness under N₂ gas and weighed. From here on, combined fractions 46/47 will be referred to as KmTx 1, and fraction 36 will be referred to as KmTx 3.

LC₅₀'s were calculated from a dilution series of KmTx 1, KmTx 3, saponin, and a sonicated suspension of K. micrum (CCMP 1974) (5 ml; 1.2×10⁵ cells ml⁻¹). LC₅₀values and ranges were determined by Probit analysis (SPSS Base 10.0, SPSS Inc., Chicago Ill.).

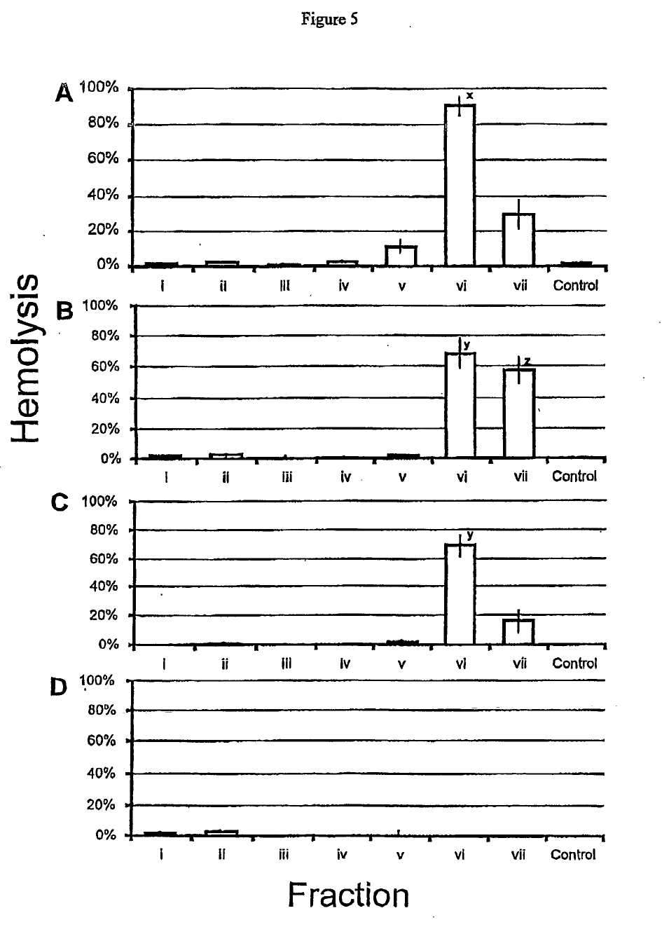

In the lipid class separation from K. micrum (CCMP 1974) cells, hemolytic activity (>90%) was observed in aliquots of fraction (vi.) (=4.78×10⁷ cells ml⁻¹) (eluted with 1:1 chloroform/methanol), which co-eluted with phosphatidylcholine (PC) according to Yongmanitchi and Ward (1992). Additional hemolytic activity (ca. 30%) was detected in fraction (vii.) (eluted with 100% methanol), co-eluting with lysophospholipid (LC) (FIG. 5 A). No hemolytic activity was observed in aliquots of separated lipid classes from P. minimum North Carolina isolate) (=6.70×10⁷ cells ml⁻¹).

Ichthyotoxicity testing using zebrafish (Danio rerio) larvae showed 100% mortality (15 of 15 larvae) in fraction (vi.) (PC) after 18 hours. Epithelial damage to pectoral and caudal fin tissue was evident at 2 hours post-exposure. No effects were observed in separated lipid class exposures from P. minimum (North Carolina isolate).

In aliquots of autotrophic and mixotrophically grown K. micrum (CCMP 1974) separated lipid classes (=1.55×10⁷ and 1.0×10⁷ cells ml⁻¹, respectively) hemolytic activity was again observed mainly in fraction (vi.) (PC). Some hemolytic activity was also observed in fraction (vii.) (LC) for both autotrophic culture lipids (ca. 60%) and mixotrophic culture lipids (ca. 30%) (FIGS. 5D and C). No hemolytic activity was observed in aliquots of separated lipid classes from Storeatula major (strain g) (=3.44×10⁷ cells ml⁻¹).

No mortality of zebrafish larvae occurred in 48 hours due to exposure to separated lipid classes from K. micrum (CCMP 1974) or S. major (strain g), in the autotrophic and mixotrophic culture experiments. However, pronounced epithelial damage was observed in 100% (15 of 15 larvae) of zebrafish exposed to aliquots of autotrophic and mixotrophic culture-derived fraction (vi.) (PC) (=1.55×10⁷ and 1.0×10⁷ cells ml⁻¹, respectively). Similar epithelial effects, although to a lesser degree, were observed in ca. 50% (7 of 15 larvae) of zebrafish exposed to aliquots of fraction (vii.) (LC) from K. micrum grown autotrophically only (=1.55×10⁷ cells ml⁻¹). Zebrafish larvae exposed to aliquots of separated lipid classes from S. major (=3.55×10⁷ cells ml⁻¹) showed no gross damage to epithelial tissues (FIG. 5D). All fish possessed a strong heartbeat and visible peripheral circulation during these exposures.

Hemolytic activity was present in the 70% methanol extract of filtered K. micrum (CCMP 1974) cells (=5.5×10⁴ cells ml⁻¹). This activity was retained in the aq-MeOH extract, even following hexane or CH₂Cl₂ washes. This activity was also present in the K. micrum (CCMP 1974) culture filtrate after passage through the type GF/F filter, but not after passage through the Sep-Pak Plus tC₁₈ cartridge. In the first culture filtrate extraction experiment (400 ml; 5.5×10⁴ K. micrum cells ml⁻¹) elution from the Sep-Pak Plus tC₁₈ cartridge resulted in hemolytic activity only in the final 100% methanol elution, while in the second experiment (2 L; 3.0×10⁴ K. micrum cells ml⁻¹) hemolytic activity was found only in the 80% methanol elution.

After washing with hexane and CH₂Cl₂, the aq-MeOH extract of cells from a K. micrum culture (400 ml; 5.5×10⁴ cells ml⁻¹) yielded 6.1 mg of organic material, whereas only 0.7 mg of material was obtained from the culture filtrate. On the other hand, five times more hemolytic activity was present in the extract from the culture filtrate.

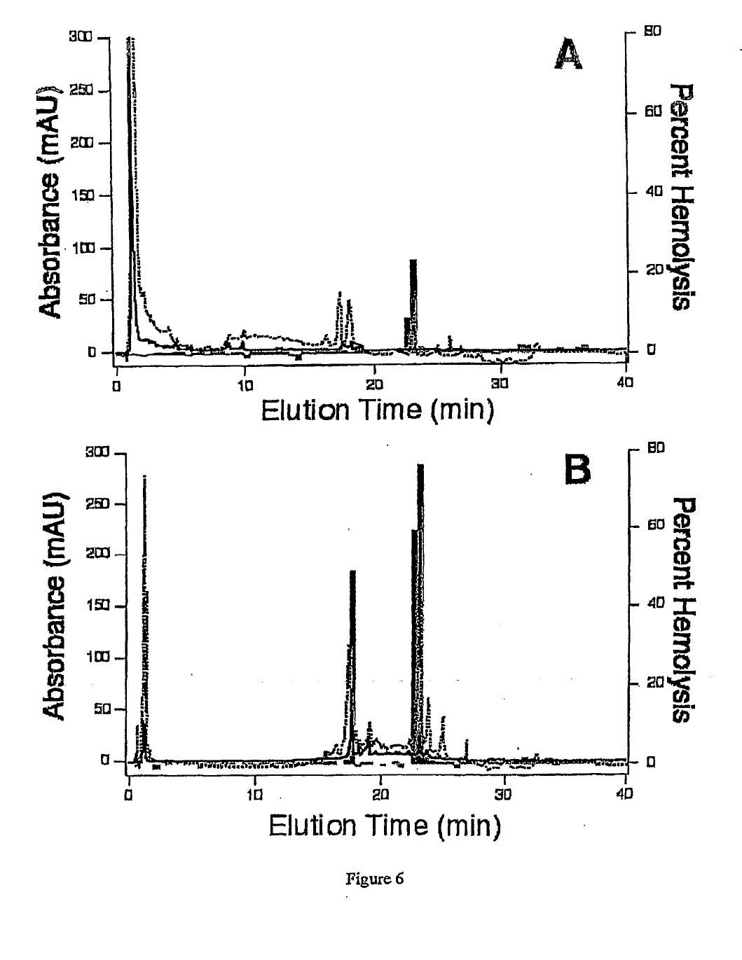

Reversed phase HPLC separation and subsequent hemolytic testing of the concentrated extracts from both the cellular and culture filtrate portions of the K. micrum (CCMP 1974) cultures yielded hemolytic activity associated with a prominent peak at ca. 23 min. (KmTx 1) (FIG. 6A). This peak was observable at 230 nm but not at 254 nm. A second peak in hemolytic activity was found only in the extracts derived from culture filtrates. This second peak eluted at ca. 17.5 min. (KmTx 3) and could be detected at both 230 nm and 254 nm (FIG. 6B). Based on UV spectrum data, these two toxins appear to be distinct compounds.

Reversed phase HPLC analysis of fractions (vi.) and (vii.) from the lipid class separation experiments from autotrophic and mixotrophically grown K. micrum (CCMP 1974) revealed that for both fractions the hemolytic activity was associated with a similar peak eluting at ca. 23 min. This peak was also observable at 230 nm but not at 254 nm. According to peak area, the material in this fraction was ca. 100 fold less than the peak obtained from the culture filtrate fractions [290 mAU for fraction (vi.) and 140 mAU for fraction (vii.) compared to 27,000 mAU from the Cl₈ extraction of culture filtrates].

Dilution series of sonicated K. micrum (CCMP 1974) cultures, as well as hemolytic HPLC fractions, responded in the hemolysis assay in a typical dose dependant manner (FIG. 4). Probit analysis from these dilution series revealed that the compound eluting at 23 min. (KmTx 1) had an LC₅₀ for the lysis of rainbow trout erythrocytes of 284 ng ml⁻¹ (range 128-744 ng ml⁻¹). Likewise, the compound eluting at 17.5 min. (KmTx 3) had an LC₅₀ of 600 ng ml⁻¹ (range 287-2864 ng ml⁻¹) The LC₅₀ for the standard hemolysin saponin was 3203 ng ml⁻¹ (range 1836-4693 ng ml⁻¹). Finally, the LC₅₀ for sonicated K. micrum (CCMP 1974) cell concentration (original culture =1.2×10⁵ cells ml⁻¹) was 2.4×10⁴ cells ml⁻¹ (range 8.4×10³-7.9×10⁴ cells ml⁻¹ ).

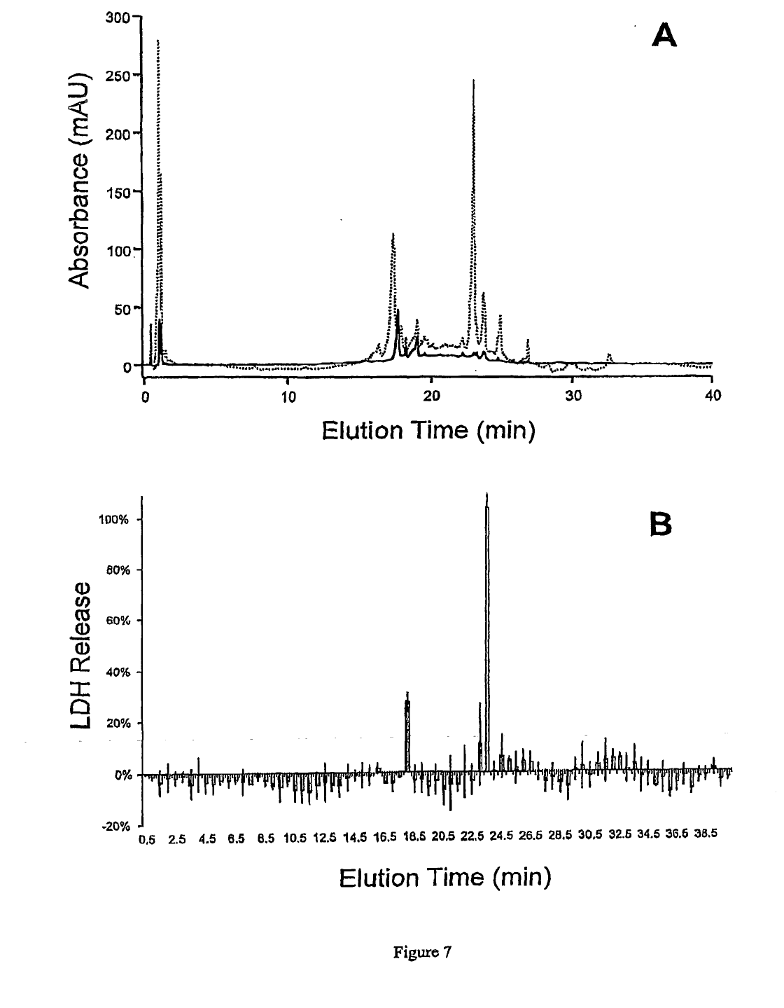

In-vitro cytotoxicity testing of aliquots of the 80 HPLC fractions (=6.0×0⁵ K. micrum cells ml⁻¹ ea⁻¹) showed toxic activity in fractions 47 (KmTx 1) (equivalent to 2000 ng ml⁻¹) and 36 (KmTx 3) (equivalent to 600 ng ml⁻¹ ) (FIGS. 7A and B). Ichthyotoxic testing of aliquots of KmTx 1 and KmTx 3 (=2.4×10⁵ cells ml⁻¹ ea⁻¹ ) showed 80% mortality (12 of 15 larvae) of zebrafish after 24 hours due to exposure to KmTx 1 only (800 ng ml⁻¹ ). KmTx 3 (250 ng ml⁻¹ ) showed no ichthyotoxic activity. Ichthyotoxic effects did not change after 48 hours of exposure.

Dinoflagellate Lysis due to Copper Sulfate and Potassium Permanganate

Isolates of both Karlodinium micrum (CCMP 1974) and Prorocentrum minimum (strain PM-1) were exposed to either CuSO₄·5 H₂O (0.5, 2, or 8 mg L⁻¹ CuSO₄) [=0.2, 0.8, 3.18 mg L⁻¹ Cu], or KMnO₄ (2, 4, or 16 mg L⁻¹ ). Mid-range values of CuSO₄ and KMnO₄ (2 mg L⁻¹ and 4 mg L⁻¹, respectively) approximated dosages typically applied at HyRock Fish Farm, while high range values (8 mg L⁻¹ CuSO₄ and 16 mg L⁻¹ KMnO₄) approximated published LC₅₀ values for aquaculture species (Tucker, 1987; Reardon and Harrell, 1990). Cell lysis was evaluated using the methods previously described.

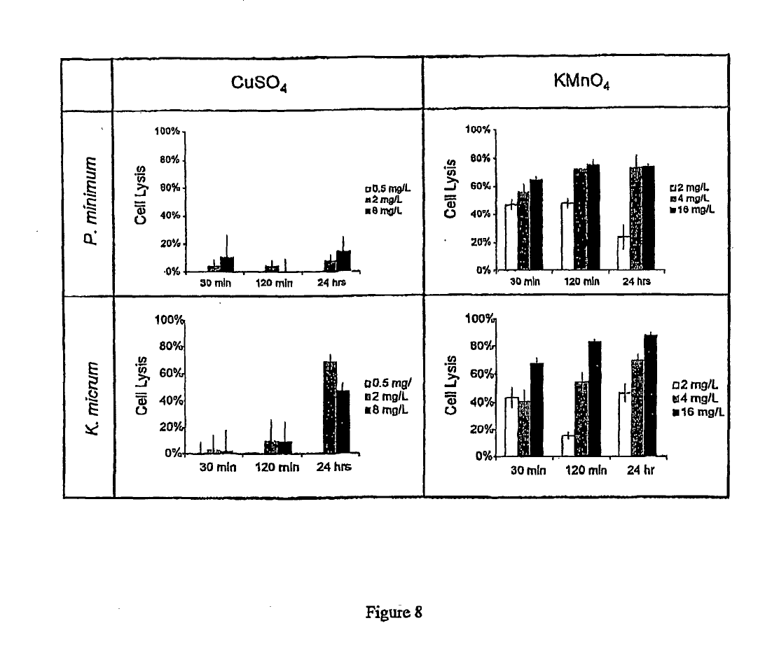

Cell Lysis (range 15% -90%) occurred in both Karlodinium micrum (CCMP 1974) and Prorocentrum minimum (strain PM-1) cultures upon exposure to KMnO₄ at all concentrations and times tested (FIG. 8). Cell lysis due to CuSO₄ exposure occurred in K. micrum, but not in P. minimum, cultures at exposures ≧2 mg L⁻¹ and only after 2 hours (FIG. 8). Microscopic observation confirmed that copper exposed K. micrum cells were swelling and lysing. In both algicidal exposures, swimming dinoflagellates were typically observed after 24 hours in low treatments only (0.5 mg L⁻¹ CuSO₄ and 2 mg L⁻¹ KMnO₄).

Toxic Activity of Copper Sulfate and Potassium Permanganate Treated Dinoflagellate Cultures and Culture Fractions

Karlodinium micrum (CCMP 1974), Prorocentrum minimum (strain PM-1), and Pfiesteria piscicida (CCMP 1921) cultures (1.5-2.5×10⁵ cells ml⁻¹ ) were exposed in 6 well polystyrene non-tissue culture treated plates (Becton Dickinson Labware, Franklin Lakes N.J.) to either CuSO₄·5 H₂O (2 mg L¹ CuSO₄) [=0.8 mg L-¹ Cu] or KMnO₄ (4 mg L-¹) and assayed for hemolytic activity. Controls consisted of untreated cultures of the same density. Analysis of variance with Scheffe's F post-hoc test was used to test for statistically significant (p<0.05) differences among treatments (StatView 4.5, Abacus Concepts Inc., Berkley Calif.).

To validate that hemolytic activity observed in Cu treated K. micrum cultures was not due to exogenous free Cu, two additional experiments were performed. The first experiment involved the addition of EDTA (2 nM) [as EDTA +4 Na·2 H₂O ] to 0.9% NaCl solutions containing CuSO₄·5 H₂O (0.1, 0.5, 2.5, or 10 mg L⁻¹ Cu). Free Cu was measured using the porphyrin method (range 0-210 μg L⁻¹ ) (HACH, Loveland Colo.). An accuracy check, according to the manufacturer's recommendations, was within acceptable limits. Each solution, with and without EDTA, was assayed for hemolytic activity. The second experiment involved exposing cultures of K. micrum (CCMP 1974) and P. minimum (Maryland and North Carolina isolates), all 1.5×10⁵ cells ml⁻¹, to CuSO₄·5 H₂O(2 mg L⁻¹ CuSO₄) then testing for hemolytic activity with and without the subsequent addition of EDTA (2 mM).

Ichthyotoxic activity was assayed by adding 3-sheepshead minnow larvae (<24 hour old post-hatch) to K. micrum (CCMP 1974) culture (2 ml; 1.5×10⁵ cells ml⁻¹ ) exposed to the same Cu and KMnO₄ treatments described above. This experiment was run in triplicate. For these experiments, 12 psu ASW (Instant Ocean Brand) with added f/2-Si nutrient mixture (Guillard, 1975) and 1.5% soil extract and 0.3% chicken manure extract (alkalinity 75 mg L⁻¹ CaCO₃) was used as the diluent. Controls were run exposing sheepshead minnow larvae to CuSO₄ or KMnO₄ alone.

An additional set of experiments was run in which aliquots of both the hemolytic methanolic extracts, isolated from both the K. micrum cells and from the culture filtrate, and aliquots of KmTx 1 and KmTx 3, were diluted back to a cellular equivalent of 5.5×10⁴ cells ml⁻¹ for the methanolic cellular extracts, and 3.0×10⁵ cells ml⁻¹ for the HPLC fractions, using filter sterilized (0.22 μm) culture media as the diluent, and mixed with either CuSO₄·5 H₂O (2 mg L⁻¹ CuSO₄), CuSO₄·5 H₂O (2 mg L⁻¹ CuSO₄) with the subsequent addition of EDTA (2 mM), or KMnO₄ (4 mg L⁻¹ ). Each was then tested again for hemolytic activity as previously described.

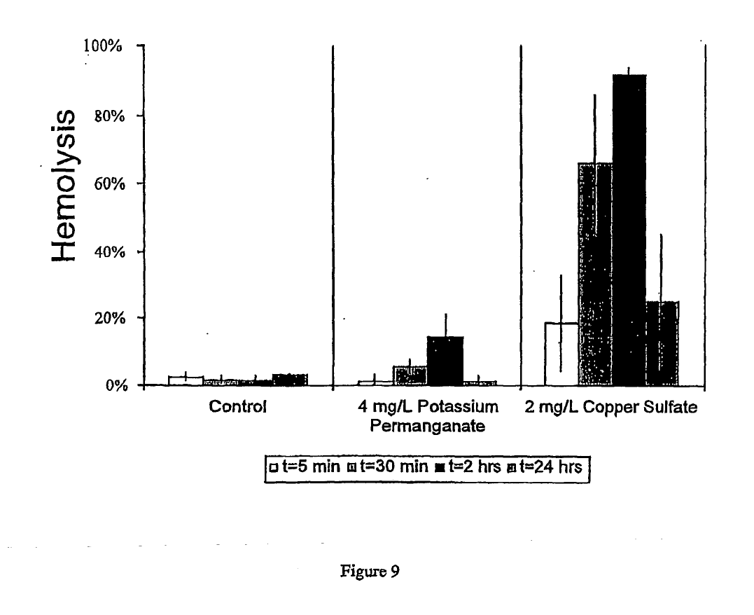

Hemolytic activity was significantly greater in Karlodinium micrum (CCMP 1974) cultures (2.5×10⁵ cells ml⁻¹) exposed to CuSO₄ (2 mg L⁻¹) compared to controls and to cultures exposed to KMnO₄ (4 mg L⁻¹) p<0.0001) at 30 min. and 2 hours, but not at 5 min. and 24 hours, using ANOVA with Scheffe's F post-hoc test (FIG. 9). No hemolytic activity was observed in cultures of Prorocentrum minimum (North Carolina and Maryland isolates) or in cultures of Pfiesteria piscicida (CCMP 1921), all 1.5×10⁵ cells ml⁻¹, exposed to either CuSO₄ or KMnO_{4.}

In EDTA/Cu chelation experiments, EDTA (2 mM) was shown to chelate >90% of free Cu (range 90.4% -96.3%) at all concentrations tested and shown below in Table 1.

TABLE 1
Copper chelation experiments measuring free copper in solution after the addition
of EDTA (2 mM).
Measured using porphyrin method, n = 2.

| | MEASURED AMOUNT (mg L⁻¹) | | | | |
|---|---|---|---|---|---|
| | Copper | | Copper + EDTA (2 mM) | | |
| | AVG | Range | AVG | Range | % DECREASE |
| 0.2 | 0.197 | 0.196-0.197 | 0.0073 | 0.0071-0.0075 | 96.3 |
| 1 | 1.034 | 1.011-1.057 | 0.042 | 0.032-0.053 | 95.9 |
| 5 | 4.957* | 4.940-4.975 | 0.477 | 0.440-0.515 | 90.4 |
| 20 | 21.110* | 20.760-21.460 | 1.570 | 1.560-1.580 | 92.6 |

*Indicates presence of hemolytic activity when mixed with an equal volume of a 5% rainbow trout erythrocyte suspension.

The addition of EDTA was shown to completely remove hemolytic activity due to Cu alone up to the highest concentration tested (10 mg L⁻¹ ). Finally, EDTA did not significantly (P>0.05) reduce hemolytic activity in K. micrum (CCMP 1974) cultures (1.5×10⁵ cells ml⁻¹) exposed to CuSO₄ (2 mg L⁻¹).

Ichthyotoxicity to sheepshead minnow larvae was observed at 24 hours post-exposure in CuSO₄ treated, but not in KMnO₄ treated K. micrum (CCMP 1974) cultures (1.5×10⁵ cells ml⁻¹) at all concentrations tested (80% mortality, 7 of 9 larvae, in 0.5 mg L⁻¹ CuSO₄ treatment and 100% mortality, 9 of 9 larvae, in 2 and 8 mg L⁻¹ treatments). No mortality or epithelial damage was observed in CuSO₄ treated control larvae, but 100% mortality did occur at 24 hours at the highest level of KMnO4 tested (16 mg L⁻¹). Prior to mortality in copper treated K. micrum exposures, several sub-lethal effects were observed. As early as 1 hour post-exposure, severe epithelial damage to the sheepshead minnow larvae occurred in K. micrum cultures at all levels of CuSO₄ tested. These effects included epithelial sloughing, and the complete deterioration of both pectoral and caudal fin tissue. Heartbeat and peripheral circulation were still present at this time. In all KMnO₄ control exposures, an orange flocculent was present which increased in abundance with increasing KMnO₄ concentration. Except for the 16 mg L⁻¹ treatment, this did not result in any mortality. Sheepshead minnow larvae exposed to control (untreated) K. micrum cultures showed no mortality or epithelial effects. These results did not change after 96 hours of exposure.

The addition of either CuSO₄ (2 mg L⁻¹), CuSO₄ (2 mg L⁻¹) with the subsequent addition of EDTA (2 mM), or KMnO₄ (4 mg L⁻¹) to hemolytic extracts from both the K. micrum (CCMP 1974) cells and culture filtrates, as well as to hemolytic HPLC fractions KmTx 1 and KmTx 3, resulted in the complete disappearance of hemolytic activity in extracts exposed to KMnO₄ only. The addition of CuSO₄, both with and without the subsequent addition of EDTA, resulted in no significant, (p>0.05) change in hemolytic activity compared to untreated hemolytic extracts as shown below in Table 1.

It was shown that dinoflagellate cell disruption, leading to release of the compound(s), followed by toxic activity, is promoted through copper sulfate (CuSO₄) treatment, while potassium permanganate (KMnO₄) treatment causes cell disruption with no subsequent toxic effects (Table 2).

TABLE 2
Summary of toxic activity from cultures of Karlodinium micrum.

| | | | | HPLC | |
|---|---|---|---|---|---|
| | WHOLE CULTURES | | | FRACTIONS | |
| TOXICITY | | Cu | KMnO₄ | KmTx | KmTx |
| ASSAY | Sonicated | Treated | Treated | 1 | 3 |
| Ichthyotoxicity | + | + | − | + | − |
| Cytotoxicity* | n.a. | n.a. | n.a. | + | + |
| Hemolytic Activity | + | + | − | + | + |

*Cytotoxicity assay was not available for testing whole dinoflagellate cultures due to incompatibilities in culture media requirements between mammalian cells and dinoflagellates.

Karlodinium micrum has been shown to be an important component of the phytoplankton community in both the Maryland and Virginia portions of the Chesapeake Bay (Marshall, 1999; Li et al., 2000). In the Chesapeake Bay, Li et al. (2000) found that K. micrum reached maximum densities ca. 4×10³ cells ml⁻¹ in the main-stem of the mid to upper Bay during late spring and early summer, often dominating the 2-20 μm photosynthetic nanoflagellate community. These densities are below those typically associated with fish mortalities, but surface densities ranging from 6×10⁴ to 2×10⁵ cells ml⁻¹ have been observed at HyRock Fish Farm during blooms both in ponds and in a channel of the Manokin River from which the farm draws water.

As mentioned previously, Pfiesteria piscicida was shown to be present at HyRock Fish Farm during the first kill in 1996 (ca. 300 cells ml⁻¹). Because of its common co-occurrence in nature and its similarity in appearance to P. piscicida under light microscopic examination, K. micrum has been grouped, along with Cryptoperidiniopsis sp., into the category of “Pfiesteria-like organisms” (PLOs) (Marshall, 1999). P. piscicida has been implicated as the causative agent in numerous fish kills in Mid-Atlantic and southeastern U.S. estuaries (see Burkholder and Glasgow, 1997), therefore its involvement in the kills at HyRock cannot be ruled out. In the current study, both P. piscicida (CCMP 1921) and Cryptoperidiniopsis sp., both grown on algae, were not found to produce any hemolytic substances up to densities of 1.5×10⁵ cells ml⁻¹. Glasgow et al. (1998) observed that P. piscicida does feed on fish erythrocytes, but during the one hour incubation in this study measurable amounts of hemoglobin were not released. These findings still do not rule out the possible involvement of P. piscicida in the fish kills at HyRock considering “toxic” and “non-toxic” varieties have been reported (Burkholder et al., 2000; Burkholder et al. 2001). But, considering the repeated associations between high K. micrum cell numbers and fish mortality and now the laboratory confirmation of toxic material production with properties consistent with observed effects during kills, K. micrum does appear to have been a contributing factor to the observed fish mortalities at HyRock Fish Farm making it a new management concern for the estuarine aquaculture industry.

In the current study, all ichthyotoxic fractions were found to have a generalized necrotic effect on the epidermis of larval zebrafish (Danio redo) and sheepshead minnows (Cyprinodon variegatus), typically starting with the pectoral and caudal fins, but not initially affecting heartbeat or peripheral circulation. More detailed studies on the effects of K. micrum and its toxin(s) on fish gills are currently being performed.

It was observed that the cellular equivalent of material required to cause lysis of rainbow trout erythrocytes was ca. 100 fold higher using hemolytic materials obtained from the lipid extraction procedure compared to sonicated cell suspensions alone (10⁷ compared to mid 10⁴-10⁵ cells ml⁻¹, respectively). It was later confirmed through reversed phase HPLC analysis that the more prominent of the two toxic fractions, KmTx 1, was in fact found in ca. 100 fold lower amounts in equivalent injections of extracts obtained through the lipid class separation compared to the C₁₈ culture filtrate procedure (based on peak area, 290 mAU from fraction (vi.) (PC) and 140 mAU from fraction (vii.) (LC) compared to 27,000 mAU from the C₁₈ extraction of culture filtrates). Therefore, it was determined that extraction procedures targeting cellular lipids, using silica columns, are not the most efficient means of toxin isolation. In the C₁₈ column extraction of culture filtrates, five times more hemolytic activity was present in nine times less total material, suggesting that the isolation of toxic materials from the culture filtrate using C₁₈ columns is the most efficient method among those tested in this study.

The hemolytic activity in the aq-MeOH extract of K. micrum cells did not partition into either hexane or methylene chloride indicating that the toxins behave like polar-lipids. Reversed phase HPLC separation of extracts from both the K. micrum cellular fraction and culture filtrates, as well from the hemolytic lipid fractions (vi.) (PC) and (vii.) (LC) from the initial lipid class separation, showed that the main peak in toxic activity corresponded to an elution time of ca. 23 min. (see FIG. 6). This suggests that the same compound was isolated in all three procedures. Using material extracted from the culture filtrate, this peak was further shown to be both ichthyotoxic and cytotoxic. On a per weight basis this compound is 10 times as potent as the standard hemolysin saponin.

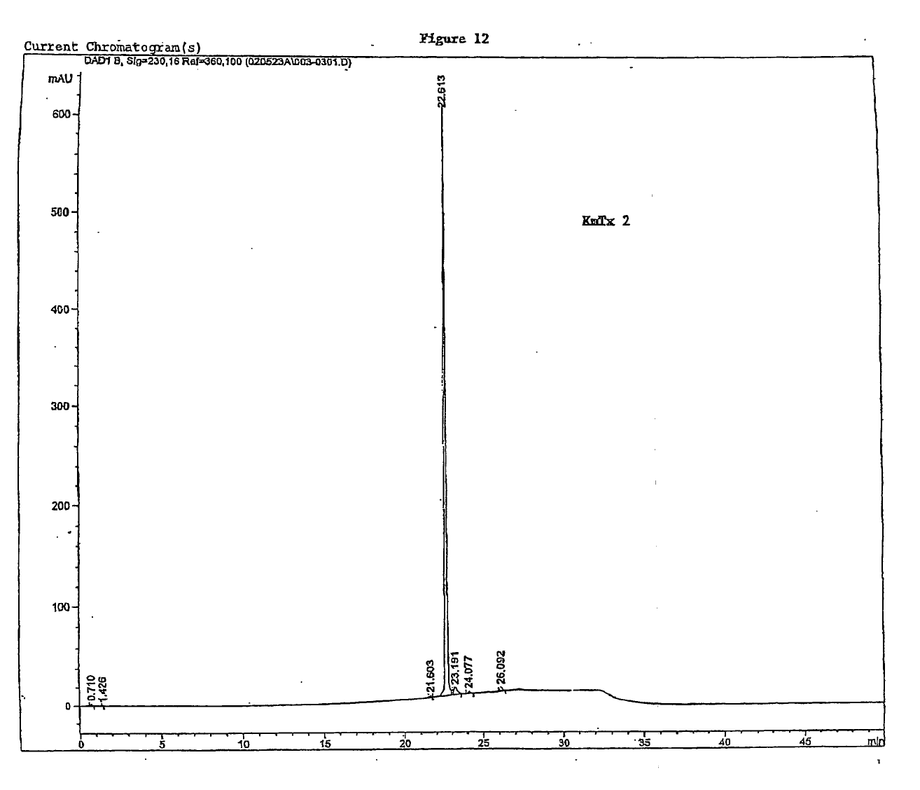

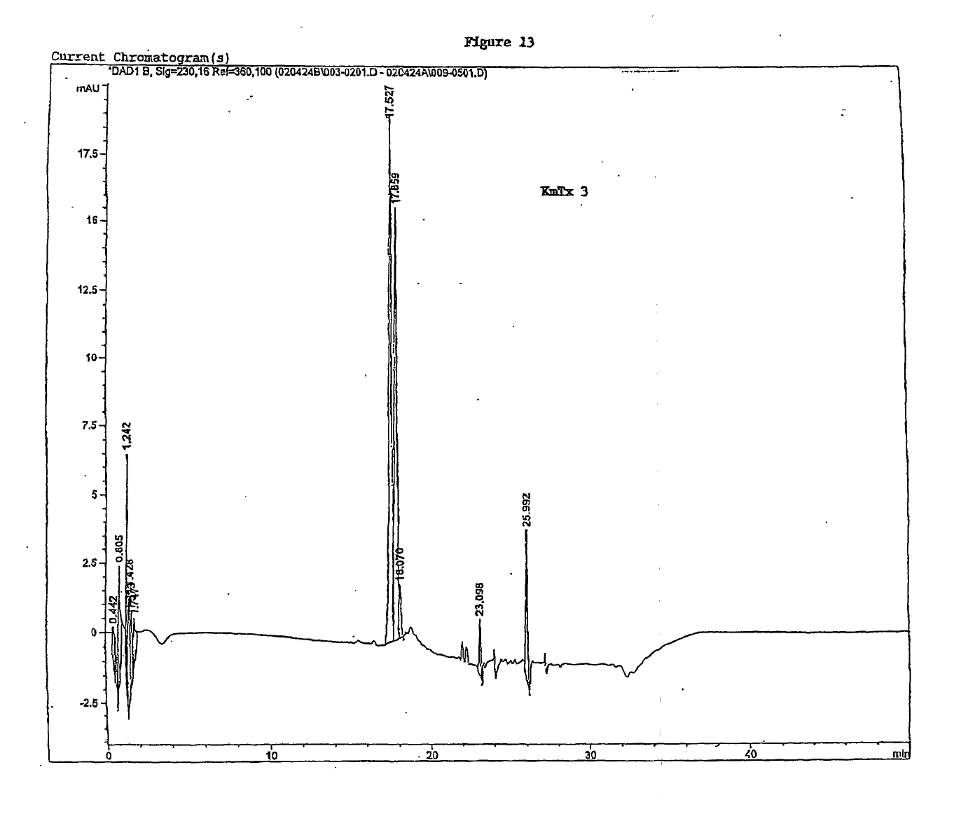

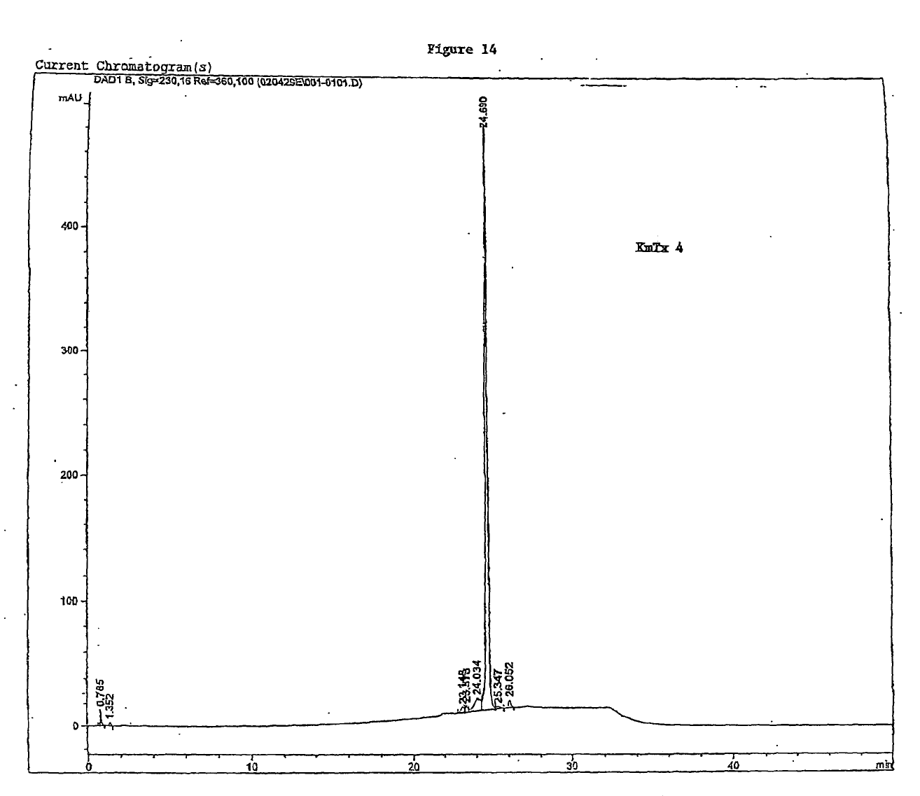

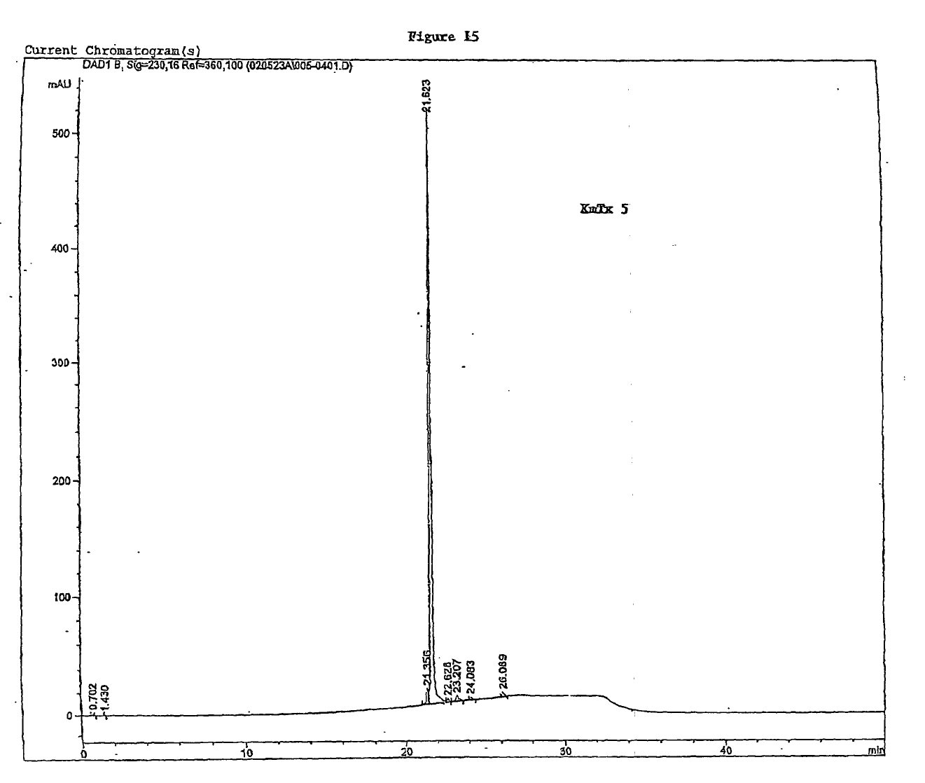

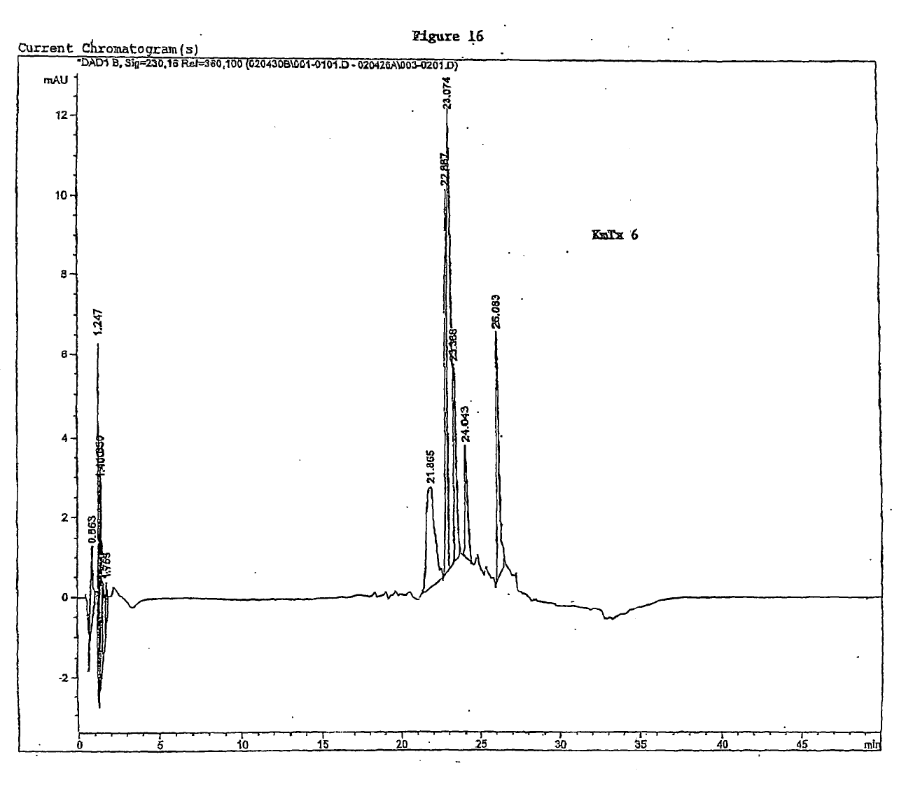

A second peak in hemolytic activity (KmTx 3), eluting at ca. 17.5 min., was found in the materials isolated from the culture filtrate only. This peak was further shown to be cytotoxic, but not ichthyotoxic, and was 5 times as potent as saponin. Further peaks similar to the peaks eluted at approximately 23 and 17.5 are shown in FIGS. 12 to 16.

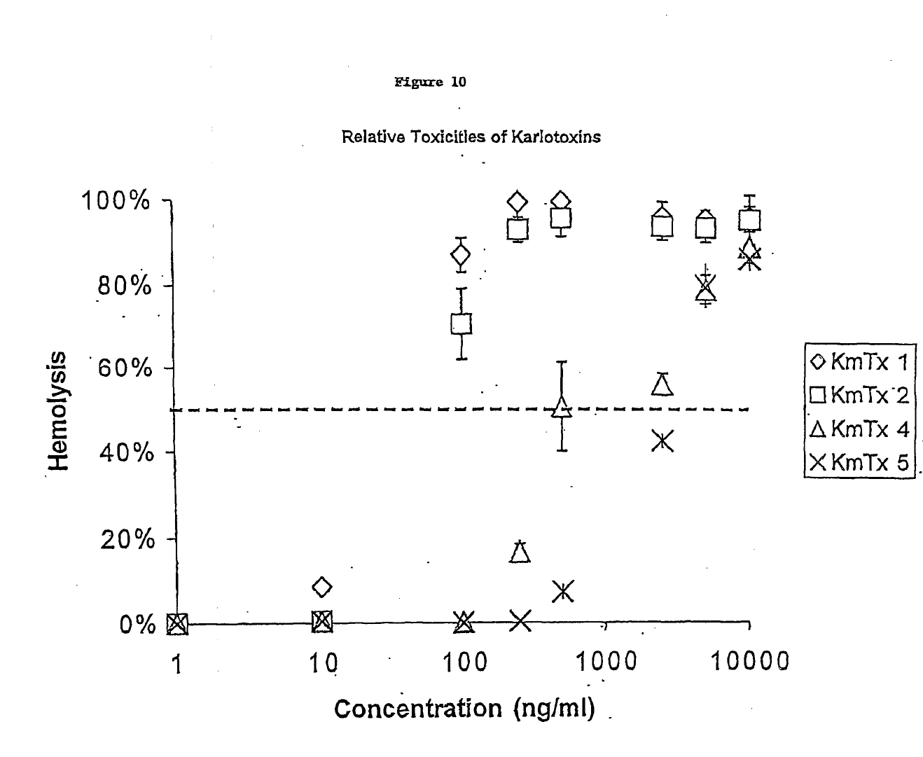

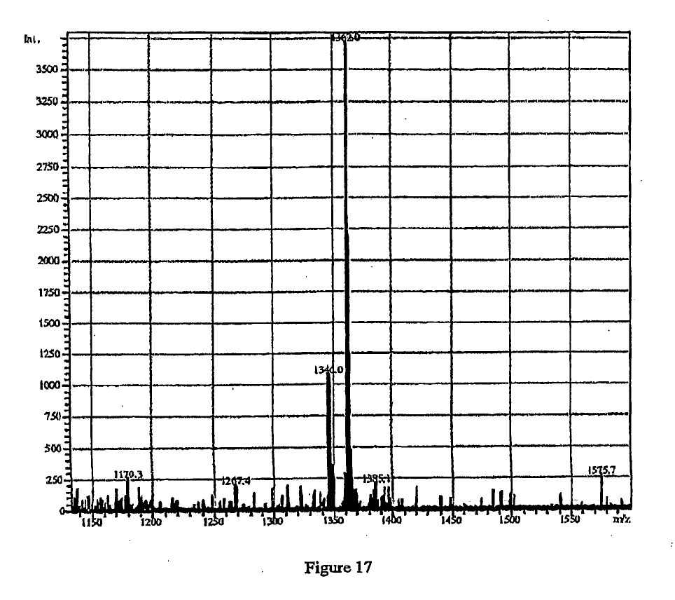

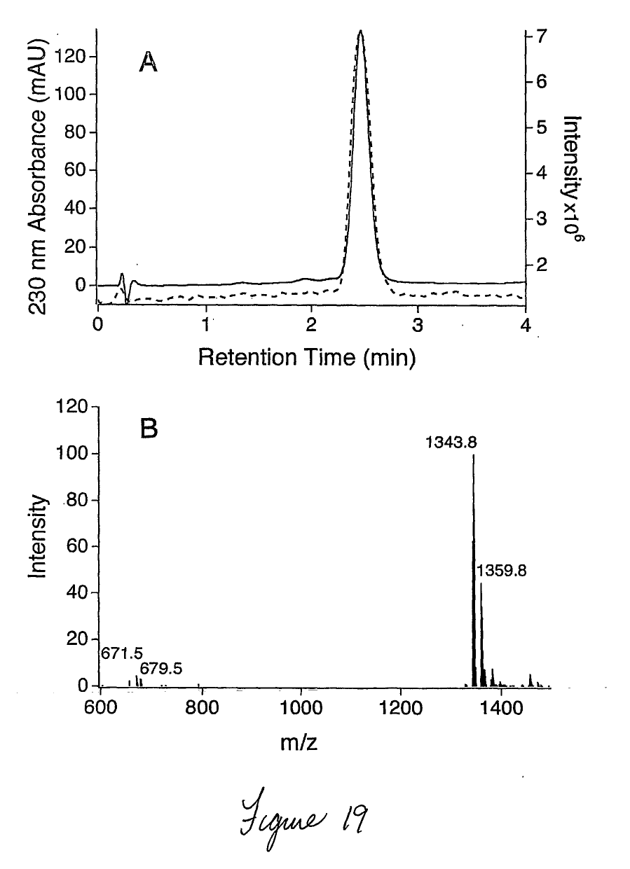

The hemolytic activity profiles of KmTx 1, KmTx 2, KmTx4 and KmTx 5 are shown in FIG. 10. The Mass Spectra of KmTx1 and KmTx 2 was determined and illustrated in FIGS. 17, and 19, respectively, and showing a molecular mass of 1362 and 1344 daltons, respectively.

Management of Algicidal Treatments

Few algicidal treatments are available to the aquaculture industry, due mainly to concerns over both expense and the regulatory issues involved with the use of harmful chemical substances with food fish. In this study, two of the more common treatments for the control of phytoplankton abundance in aquaculture ponds, copper sulfate and potassium permanganate, were examined.

Costing ca. $25.00 to treat one 5 acre pond (avg. depth 5 ft.), copper sulfate is one of the most commonly used chemicals for the control of both noxious weed species and infectious diseases in fish ponds and hatcheries (Boyd, 1990; Masser, 2000) (A. Mazzaccaro, HyRock Fish Farm, personal communication). Application rates for copper sulfate at HyRock typically ranged from 1-2 mg L⁻¹, well below the experimentally determined 96h LC₅₀ of ca. 8 mg L⁻¹ determined for striped bass fingerlings at comparable salinities, although the study referred to was performed at higher alkalinities than those typically found at HyRock Fish Farm (Reardon and Harrell, 1990). Regardless, prior to the events of Jul. 30, 1996, this application dosage had been used previously at HyRock, at the same alkalinities present during the 1996 fish kill, to control green algal blooms without difficulty.

Potassium permanganate has been used in aquaculture facilities for various reasons ranging from disease and external parasite treatment to the oxidation of organic and inorganic substances to reduce both biological and chemical oxygen demands (Tucker and Boyd, 1977; Tucker, 1987; Tucker, 1989; Boyd, 1990; Noga, 2000). An added benefit of algicidal KMnO4 treatment is that reduction of KMnO₄ yields manganese dioxide (MnO₂) which forms a colloid with an outer layer of exposed OH groups. These groups are capable of adsorbing both charged and neutral particles from the water column, thereby further promoting the precipitation of microorganisms (Environmental Protection Agency, 1999). The 96h LC₅₀ for KMnO₄ was shown to range from 4.5 to 17.6 mg L⁻¹ for channel catfish fingerlings depending on the amount of dissolved organic material in the system (Tucker, 1987). HyRock, like any confined animal feeding operation (see Glibert and Terlizzi, 1999) tends to possess very high organic loads.

A simple method for determining the required dosage in such an environment is the 15-min. KMnO₄ demand in which the concentration of KMnO₄ required to color the water for 15 min. is multiplied by 2.5 to determine the application rate (Tucker, 1989). In the current study, the 15-min KMnO₄ demand for the culture media in which the controlled exposures were performed was ca. 2 mg L⁻¹, due to the added soil and chicken manure extracts, making the recommended dosage ca. 4-5 mg L⁻¹. At HyRock, the typical application was <4 mg L⁻¹, actually below recommended, due mainly to cost constraints. Treatment of one 5 acre pond (avg. depth 5 ft.) costs ca. $500.00 (A. Mazzaccaro, HyRock Fish Farm, personal communication).

In the current study, copper sulfate was shown to lyse K. micrum cells, within the range of dosages used at HyRock, and in doing so promoted the release of toxic substance(s). Potassium permanganate was shown also to lyse K. micrum cells within the range of dosages applied at HyRock, but at the same time, was also shown to remove the toxic activity from whole cultures, from cell and filtrate extracts, as well as from HPLC fractions of KmTx 1 and KmTx 3. Despite the vast difference in treatment cost, based on this data, potassium permanganate is recommended over copper sulfate for the treatment of K. micrum blooms in aquaculture facilities.

Aquaculture currently represents about 20% of world fisheries production, and this number is only projected to increase in the future (Botsford et al., 1997; Boyd, 1999). The results of this study have shown that Karlodinium micrum produces at least one compound which is hemolytic, ichthyotoxic and cytotoxic. Although this organism has not historically been associated with aquatic faunal mortalities in the main stem of the Chesapeake Bay it has been observed in sufficient numbers in mid-salinity tributaries to be a new management concern.

### EXAMPLE 3

Blood was extracted from the caudal vein of rainbow trout (Oncorhynchus mykis). Erythrocyte suspensions were prepared by washing three times (2000 g for 5 min.) with ice-cold buffer [150 mM NaCl, 3.2 mM KCl, 1.25 mM MgSO₄, and 12.2 mM Tris base]. Buffer pH was adjusted to 7.4 at 10° C. with 1N HCl, then filter sterilized (0.22 um). Buffy coats were carefully removed and, after the third wash, cells were stored in Tris buffer with 3.75 mM CaCl₂ at 50% of their original concentration. Blood suspensions were stored at 4° C. for no longer than 10 days.

Hemolysis assays were performed by diluting test material in Tris buFFer +CaCl₂ (100 ul) and adding this to 100 ul of a 5% erythrocyte suspension for a total assay volume of 200 ul. Assays were run in 96 well, V-bottom, non-treated, polystyrene plates (Corning Inc., Coming N.Y.) sealed with Falcon 3073 pressure sensitive film (Becton Dickinson Labware, Lincoln Park N.J.). Plates were incubated on an orbital shaker (80-100 rpm) at 20° C. for 1 hour. Plates were then centrifuged at 2000 g for 5 min. and the supernatant (100 ul) was transferred to a new flat bottom 96 well polystyrene plate, where the absorbance of released hemoglobin was read at 540 nm. Saponin (from Quillaja bark; Sigma Chemical Co., St. Louis Mo.) was used as a positive hemolysin control. Percent hemolysis (0-100%) was calculated from comparison to un-treated controls and samples treated with 10 ug saponin. All treatments were run in quadruplicate.

KmTx 2 Purification.

KmTx 2 used in this study was isolated directly from water collected during a fish kill in a South Carolina brackish pond described in Kempton et al. (2002). Previously frozen and thawed water samples (1.6L total) were first passed through type GF/F filters (Whatman International Ltd., Maidstone, England), then lipophilic materials were isolated from filtrates using several small (3 ml) disposable C₁₈ cartridges (Sep-Pak Plus tC₈, Waters Corporation, Milford, Mass.). C₁₈ cartridges were first pre-equilibrated with methanol (MeOH) then water (20 ml ea⁻¹). After 40% and 60% MeOH washing steps (15 ml ea⁻¹), hemolytic materials eluted from the cartridges with 80% MeOH (15 ml). The hemolytic 80% MeOH fraction was dried under N₂, re-suspended in a small volume of MeOH and fractionated further using HPLC. Aliquots were injected onto a LiChroDART 1254/RP8 (5 μm) reversed-phase column (Waters Corporation, Milford, Mass.) and eluted at 30° C. with a MeOH/H₂O (30:70) to (95:5) linear gradient, over 20 min, at a flow rate of 1 mi/min (Hewlett Packard Series 1100 HPLC System, Agilent Technologies, Inc. Wilmington, Del.). Fractions were collected every 0.5 min and assayed for hemolytic activity as previously described. This particular sample contained only one hemolytic peak. Molecular weight of this peak was determined through liquid chromatography/mass spectroscopy (LC/MS) analysis. To first confirm sample purity a 0.5 μg sample was separated on a Zorbax 2.1×50 mm SB-C8 column using an Agilent 1100 series HPLC system (Agilent Technologies, Wilmington, Del.), eluted at 30° C. with a MeOH/H₂O (60:80) to (80:60) linear gradient, over 6 min, at a flow rate of 0.7 ml/min. Both MeOH and H₂O contained 0.1% formic acid to facilitate sample ionization. Samples were then analyzed both in the positive and negative modes using an Agilent G1946D single quad mass spectrometer detector (Agilent Technologies, Inc., Wilmington Del.). Positive ionization yielded several prominent peaks in the 1300-1400 Da range. In the positive ionization mode, the most abundant peak was at 1345.8 Da (M+H)⁺, followed in abundance by peaks at 1361.8 [(M+H)⁺+16], 1367.8 (M+Na)⁺, and 1383.8 [(M+Na)++16]. Previous attempts at mass determination, all in the positive ionization mode, yielded a mass ion of 1367.8 Da (M+Na)⁺ (data not shown). Negative ionization yielded only prominent peaks at 1343.8 (M−H)⁺ and 1359.8 Da [(M−H)⁺+16] (FIG. 19). These data led us to the conclusion that the molecular weight of KmTx 2 was 1344.8 Da., and the molecule had a tendency to form Na adducts in the positive ionization mode. Several, less abundant, peaks were also in the samples, differing from the molecular ion by 16 Da, suggesting a difference of a single oxygen atom.

Gymnodinosterol Purification.

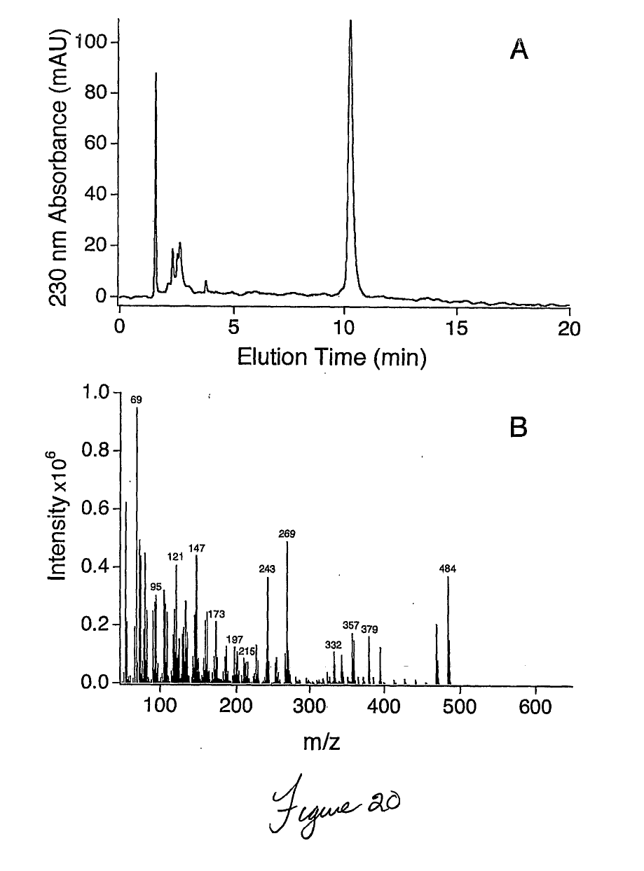

K. micrum (CCMP 2282) cells, grown as described in Deeds et al (2002), were filtered onto pre-combusted type GF/F filters (Whatman International Ltd., Maidstone, England) and extracted twice with chloroform/methanol (2:1). Extracts were concentrated under vacuum at 50° C. using a rotavapor (Buchi model R110, Switzerland) then placed in a large glass column containing 100 ml (dry volume) of Bio-Sil A (100-200 mesh) activated silica (Bio-Rad Laboratories, Richmond, Calif.) that had been pre-equilibrated with 250 ml methanol followed by 300 ml chloroform. Neutral lipids were eluted first using 250 ml chloroform, according to Yongmanitchi and Ward (1992). Neutral lipids were dried under vacuum at 50° C., then re-suspended in a small volume of chloroform. This material was then applied to a 20×20-tapered layer TLC plate (Uniplate Silica Gel G, Analtech, Newark Del.) that had been pre-developed for 2 hours with 1:1 chloroform/methanol. Next, the plates were pre-focused with 1:1 chloroform/methanol, then developed for 2.5 hrs using 250 ml of n-hexane/diethyl ether/acetic acid (80:20:1.5). After drying, several pigment bands were visible on the bottom half of the TLC plate. Additional lipid bands on the top half of the TLC plate were visualized by adding several iodine crystals to the emptied developing chamber. Four pale yellow bands became visible upon iodine vapor exposure. These 4 bands were scraped into 6 ml glass reaction chambers that contained Teflon frits (Supelco, Bellefonte Pa.). Each was eluted with 25 ml of n-hexane/diethyl ether (80:20). Fractions was then dried under vacuum at 50° C., weighted, and re-suspended in chloroform to a concentration of 5 mg/ml. To confirm which bands contained sterols, 5 μg of each sample was spotted onto activated S-III Chromarods and analyzed by thin layer chromatography/flame ionization detection (TLC/FID) on an Iatroscan TH-10 TLC/FID Analyzer (Iatro Laboratories, Tokyo, Japan). Spotted rods were pre-focused with chloroform/methanol (1:1) then developed in n-hexane/diethyl ether/formic acid (85:15:0.1) for 45 min. Bands were tentatively identified by comparison to known lipid standards. The band corresponding to sterols was dried under nitrogen, and re-suspended in a small volume of acetonitrile/methanol (1:1). The sterol-containing fraction was separated further using an Agilent 1100 series HPLC system (Hewlett Packard Corporation, Wilmington, Del.). The fraction was injected onto a STERI-5 220×4.6 mm RP-18 (1 μm) column (Applied Biosystems, Foster City, Calif.) and eluted at 51° C. with an isocratic mixture of acetonitrile/methanol/water (48.5:48.5:3). Presumptive sterol fractions were collected using cholesterol and ergosterol as standards. Collected sterol fractions were positively identified by gas chromatography mass spectroscopy (GC/MS) analysis using an Agilent 6890 Series GC with a 60 m DB5 ms, 0.32 ID, 0.25 μm film column (JW Scientific, Folsom, Calif.). Sterols were identified as trimethylsilyl ether derivatives (TMS). TMS derivatives were created by drying the samples under nitrogen then re-suspending in 1 ml of BSTFA 25% pyridine with 50 μl of TMS. This mixture was heated at 50° C. for 15 min, then re-dried under nitrogen and re-suspended in a small volume of methylene chloride. 10 μg of each sample was injected onto the GC/MS. (24S)-4α-methyl-5α-ergosta-8(14),22-dien-3β-ol (gymnodinosterol) was positively identified according to LeBlond and Chapman (2002) (approx. 80% pure) (FIG. 20).

### EXAMPLE 4

The Pfiesteria look-alike dinoflagellate Karlodinium micrum, reported as non-toxic, is shown to be just the opposite. KmTx 2, isolated from K. micrum, possesses hemolytic, cytotoxic, ichthyotoxic, and anti-fungal properties. KmTx 2 is lethal to fish at concentrations measured during fish kills, while sublethal doses damage gill epithelia. Cellular toxicity occurs through permeabilization of plasma membranes, resulting in osmotic lysis. Membrane sterol composition is an important determinant of KmTx 2 activity and appears to play a role in the immunity of K. micrum from its own toxins. This study confirms the role of K. micrum in estuarine fish kills worldwide.

Materials and Methods

KmTx 2 Isolation.

KmTx 2 used in this study was isolated from a 2 L frozen water sample collected during a fish kill that occurred in a brackish water retention pond near Charleston, S.C. on Feb. 5, 2002 described in Kempton et al. (2002) The procedures for the isolation and identification of KmTx 2 are described in Deeds et al. (2002) and Kempton et al. (2002). Tests for toxin purity and molecular weight determination are described above.

Effect of KmTx 2 on Fish.

Zebrafish (Danio rerio-60 days old) were exposed to 0.1, 0.5, 1, or 2 μg/ml KmTx 2 in 50 ml of aerated reconstituted fresh water (soft) [ASTM, 1992 #125]. Toxin dilutions were made in MeOH (200 μl max. per treatment) and controls were exposed to 200 μl MeOH only. Three fish were exposed per replicate, three replicates per treatment. Fish were observed for mortality hourly. Upon death, fish were preserved in neutral buffered formalin and prepared for histological examination as described in Noga 2000. At six hours post exposure, controls and any fish that did not die were euthanised by rapidly lowering the water temperature and prepared for histological examination as previously described.

Hemolysis Assays.

Hemolytic activity was assessed through the use of a microtiter assay utilizing rainbow trout erythrocytes, as described in Deeds et al. (2002), and detailed further above.

Osmotic Protection Assays.

The following osmolytes (all purchased from Sigma-Aldrich Co., St Louis, Mo.) were prepared as 30 mM solutions using the Tris buFFer +CaCl₂: sucrose (MW 342.3), polyethylene glycol (MW 400), polyethylene glycol (MW 600), maltohexaose (MW 990.0), polyethylene glycol (MW 8,000), dextran (MW 10,000). Osmolarity of each solution was measured using a Vapro 5520 vapor pressure osmometer (Wescor Inc., Logan Utah). Osmolarity of all solutions, with the exception of PEG 8,000 and 10,000 MW dextran, did not differ significantly from the Tris buffer (300-320 mOsm). The osmolarities of PEG 8,000 and 10,000 MW dextran were approximately double this amount (ca. 640 mOsm).

Assays were performed by preparing trout erythrocyte suspensions and toxin dilutions (0, 0.25 0.5, and 1 μg/ml) in the appropriate osmolyte solution and performing the hemolysis assay as described in supplemental materials.

Ion Permeation Measurements.

The following cell types were tested for toxicity due to KmTx 2; rat embryonic fibroblast, rat intestional epithelial, and isolated rabbit primary sensory neurons. Cells were cultured on No. 1 glass coverslips and loaded with fluorescent indicator (fura-2) and were examined on an inverted epifluorescence microscope (model Diaphot; 40×CF Fluor objective, N.A. 1.30; Nikon Corp.) coupled to a spectrofluorometer (model CM1T10I, SPEX Industries) operating in the microfluorometry mode. Cells were bathed in 4 ml of Dulbecco's modified Eagle's medium (DMEM) buffered with HEPES (pH 7.4). KmTx 2 (stock solution in DMSO) was directly bath-applied with gentle convective mixing. Fura-2 was alternately excited at 340 and 380 nm. Fluorescence emission was passed through a 510-nm bandpass filter before photometric quantitation. DATAMAX software (SPEX Industries) was used for data acquisition and instrument control. Origin software (OriginLab Corp.) was used for data reduction and analysis

Anti-Fungal Assays.

The following fungal strains were purchased from the American Type Culture Collection (Manassas, Va.), Aspergillus niger (ATCC 1004) as a representative filamentous fungi, and Candida albicans (ATCC 14053) as a representative yeast. Assays were performed according to the following National Committee for Clinical Laboratory Standards documents; M27-A2 Reference Method for Broth Dilution Antifungal Susceptibility Testing of Yeasts; Approved Standard—Second Edition([NCCLS, 2002), and M38-A Reference Method for Broth Dilution Antifungal Susceptibility Testing of Filamentous Fungi; Approved Standard (NCCLS, 2002). Procedures for the broth microdilution modification were followed for both species with the following exceptions; A. niger stocks were maintained on Potato Dextrose Agar at 25° C., while C. albicans was maintained on Yeast Malt Agar at 28° C., both according to ATCC recommendations. Amphotericin B was used as a positive control. Microdilution assays were performed in HyQ-RPMI-1640 media (HyClone, Logan, Utah.), with 2.05 mM L-glutamine, without sodium bicarbonate, pH 7.2 using dilutions of 1 mg/ml DMSO stocks of KmTx 2 and amphotericin B.

The following concentrations were tested for both KmTx2 and amphotericin B: 0.0625, 0.125, 0.25, 0.5, 1, 2, 4, 8, and 16 μg/ml. A maximum of 1.6% DMSO was added to any given well. Plates were incubated at 25 and 28° C., respectively, for A. niger and C. albicans. Plates were checked daily for growth and minimal inhibitory concentrations (MIC) were defined as the lowest dose at which no growth was observed after 96 hours. Amphotericin B and all culture media were purchased from Sigma-Aldrich (St. Louis, Mo.).

Hemolytic LC₅₀ values were calculated from a dilution series of KmTx 2 and amphotericin B. LC₅₀ values and ranges were determined by Probit analysis using a SPSS Base 10.0 statistical software package (SPSS Inc., Chicago, Ill.).

Effect of KmTx 2 on a Potential Grazer.

Oxyrrhis marina (1.7×10⁴ cells/mil) and K. micrum (CCMP 2282) (3.5×10⁴ cells/ml) were exposed, in triplicate, to 0, 0.1, 0.5, and 1 μg/ml KmTx 2 in six well non-tissue culture treated polystyrene plates (Becton Dickinson Labware, Lincoln Park N.J.). The O. marina culture was obtained through single cell isolation from a Chesapeake Bay water sample and was maintained in 15 psu artificial sea water (Instant Ocean Brand) using Rhodomonas sp. (CCMP 767) as a food source. O. marina was starved for 24 hours prior to exposures to reduce the number of food organisms. K. micrum (CCMP 2282) was maintained in 12 psu artificial sea water (Instant Ocean Brand) with f/2 nutrient mixture plus 1% soil extract as described in Deeds et al. (2002). Cell densities were measured at 1 and 24 hours using a Coulter Multisizer II particle counter by enumerating the 7-20 μm and the 15-30 μM size fractions, respectively, for K. micrum and O. marina using a Coulter Accucomp software package (Coulter Electronics Limited, Miami Fla.). Significant differences (p<0.05) among mean cell numbers for different treatments were tested for using one-way analysis of variance with Scheffe's post hoc test using SPSS Base 10.0 statistical software (SPSS Inc., Chicago, Ill.).

Hemolytic activity remaining in solution was measured at 1 hour by mixing 100 μl of each treatment with an equal volume of diluted rainbow trout RBC suspension as described above in Example 3.

Membrane Lipid Inhibition Assays.

To assess the inhibitory effects of exogenous membrane lipids on KmTx 2 hemolytic activity, the following sterols and lipids were tested: cholesterol, ergosterol, gymnodinosterol, and both natural and synthetic phosphatidylcholine. Cholesterol (5-cholesten-3β-ol) and L-α-phosphatidylcholine (brain, porcine) were purchased from Avanti Polar Lipids Inc. (Alabaster, Ala.); ergosterol (5,7,22-cholestatrien-24β-methyl-3β-ol) was purchased from Steraloids, Inc. (Newport, R.I.). Synthetic phosphatidylcholine (L-α-lecithin (β-γ-dipalmitoyl) was purchased from Calbiochem (San Diego, Calif.), Gymnodinosterol was isolated from filtered K. micrum (CCMP 2282) cells (isolation described in supplemental materials). KmTx 2: 0, 0.1, 0.5, and 1 μg/ml was added to solutions of Tris buffer +CaCl₂ with either: 0, 0.001, 0.01, 0.1, 1 or 10 μM selected sterol or membrane lipid. Stock solutions of toxin and lipid were made in methanol and no more than 1% of each (2% total) was added to any given well. After brief mixing, these solutions were added 1: 1 with a diluted suspension of rainbow trout erythrocytes and hemolytic activity was assessed as described in supplemental materials.

This study focused on the biological activities of KmTx 2, previously isolated from North Carolina, South Carolina, and Florida isolates of K. micrum. KmTx 2 is a polar lipid-like compound with a molecular weight of 1344.8 Da. The toxin used in this study was isolated directly from water collected during a South Carolina fish kill (Kempton, 2002)] in which high K. micrum densities were present.

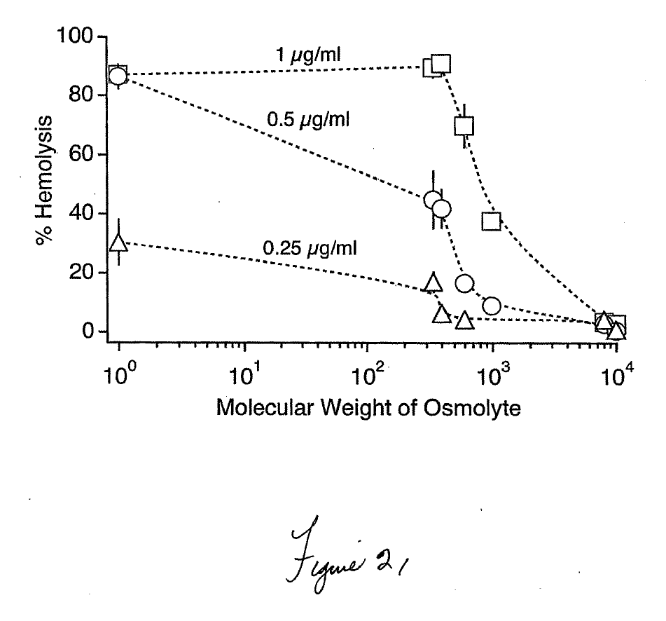

To confirm the hypothesis that KmTx 2 kills vertebrate cells by colloid osmotic lysis, we show here that co-incubation of rainbow trout erythrocytes with a range (342.3-10,000 MW) of inert osmolytes, including sugars, polyethylene glycols, and dextrans, cause a progressive inhibition of lysis with complete prevention, even at 1 μg/ml KmTx 2, at molecular weights >8,000 Da (FIG. 21). This effect was graded based on the amount of toxin used with lower inhibition at higher toxin concentrations. This data confirms the action of colloid osmotic lysis in cells exposed to KmTx 2. The use of cytolytic, pore-forming agents, is a widely used and highly successful means of cellular attack and defense. Various cytolysins are produced by organisms ranging from prokaryotes, to protozoans, to higher vertebrates. Examples of this group of compounds include glycoside saponins produced by plants, polyene-macrolide antibiotics and cytolytic proteins produced by bacteria, venoms produced by aquatic invertebrates, such as cnidarian jellyfish, and even the complement proteins of the human immune system. The presumed function of these compounds varies as well, from defense as with the saponins and complement proteins, to aids for infection and proliferation as with bacterial cytolytic proteins, to prey capture as with the jellyfish venoms. But the ultimate activity of this group of compounds is fairly consistent; permeabilization of target plasma membranes to a range of small ions and molecules resulting in an inward osmotic flux of water with subsequent cellular swelling. This process can lead to either direct osmotic lysis or a series of more complicated pathways such as endoplasmic reticulum vacuolation or apoptosis.

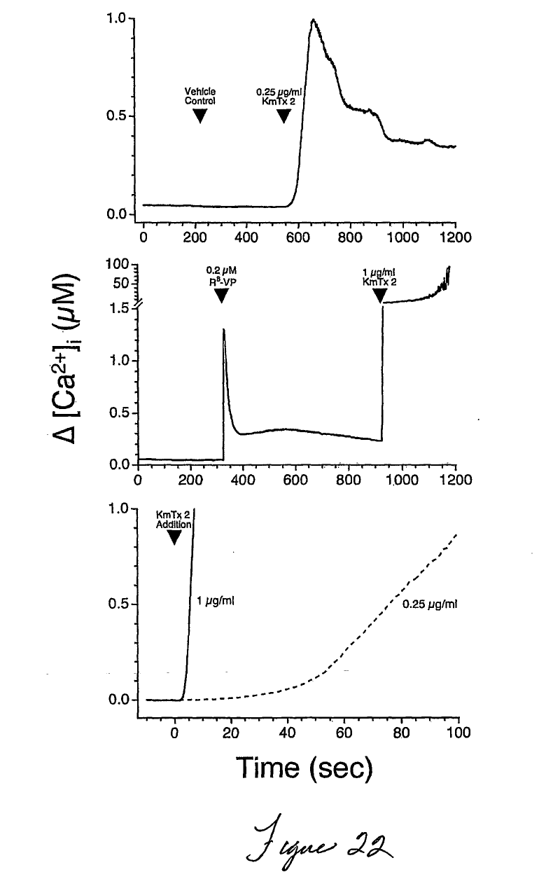

To further assess the nature of the cytotoxic activity of KmTx 2, we exposed a variety of model mammalian cell types and measured the inward flux of various cations using both intracellular fluorescent indicators and direct electrophysiological measurements. When applied to rat embryo fibroblasts at 0.25 μg/ml, KmTx 2 caused a marked increase in intracellular free Ca²⁺ concentration ([Ca²⁺]ᵢ), which then declined slowly (FIG. 22A). In contrast, at 1 μg/ml, KmTx 2 caused a sharp and irreversible rise in [Ca²⁺]ᵢ (FIG. 22B), and eventual cell lysis (not shown). The rise in [Ca²⁺]ᵢ was not observed in Ca²⁺-free medium; therefore, we infer that the toxin's action was to increase the ionic permeability of the plasma membrane. Essentially similar results were obtained when these experiments were repeated on additional cell types, including rat intestinal epithelial cells, vagal sensory neurons from adult rabbit, as well as rat ventricular cardiac myocytes. FIG. 22C shows the effect of KmTx 2 on a rat cardiac myocyte loaded with fura-2 indicator. At rest, the myocyte displayed low resting [Ca²⁺]ᵢ (pseudo-color image), and normal relaxed morphology (bright-field micrograph). 60 seconds after application of 0.25 μg/ml KmTx 2, [Ca²⁺]ᵢ was dramatically elevated, and the myocyte was irreversibly contorted by hyper-contraction. The increased membrane permeability induced by KmTx 2 was not selective for Ca²⁺. Wwe determined that KmTx 2 promoted the permeation of cations as different as Na⁺ and Mn²⁺, in addition to Ca²⁺ (data not shown).

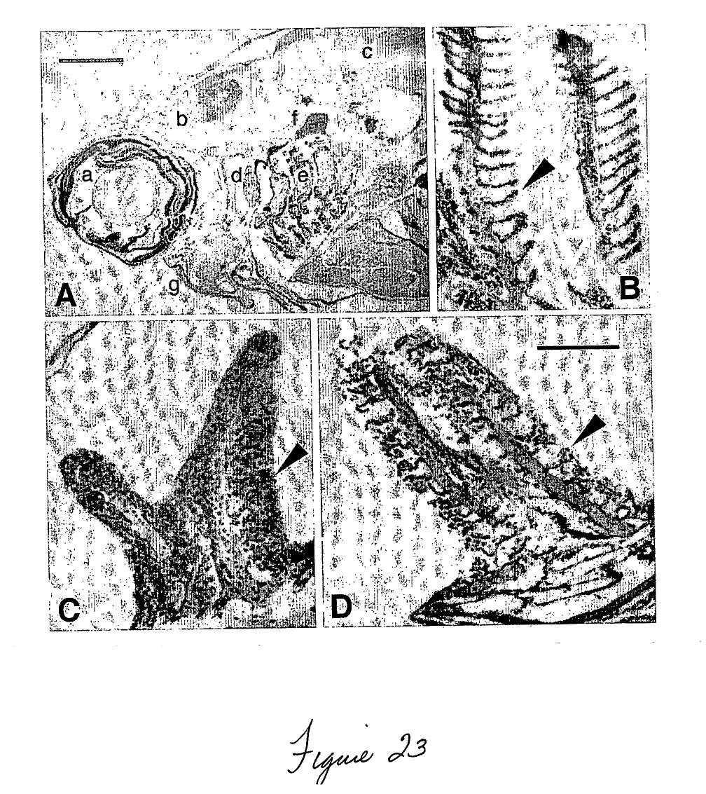

To confirm the ichthyotoxicity of KmTx 2, 60 day old zebrafish (Danio rerio) were exposed to an increasing concentration of toxin. Fish exposed to 0.5 μg/ml KmTx 2 all died in <1 hour. Fish exposed to 0.1 1μ/ml survived the full 6 hr exposure but significant mucus production was observed coming from the opercula. Histological examination of sectioned whole fish showed loss of secondary structure of gill lamellae and sloughing of gill epithelial tissue in all 0.1 μg/ml KmTx 2 treated fish (FIGS. 23 A-D). Severe necrosis and degeneration of gill tissue was observed in all 0.5 μg/ml treated fish. Excessive mucus production and some necrosis and degeneration of gut epithelial tissue was observed in all treated fish with effects being most pronounced in 0.1 μg/ml treatments (not shown). For all 0.1 μg/ml treated fish, gross histological changes were not observed in any other organs including skin, liver, kidney, heart, brain, muscle, pseudobranch, thymus, or gonads.

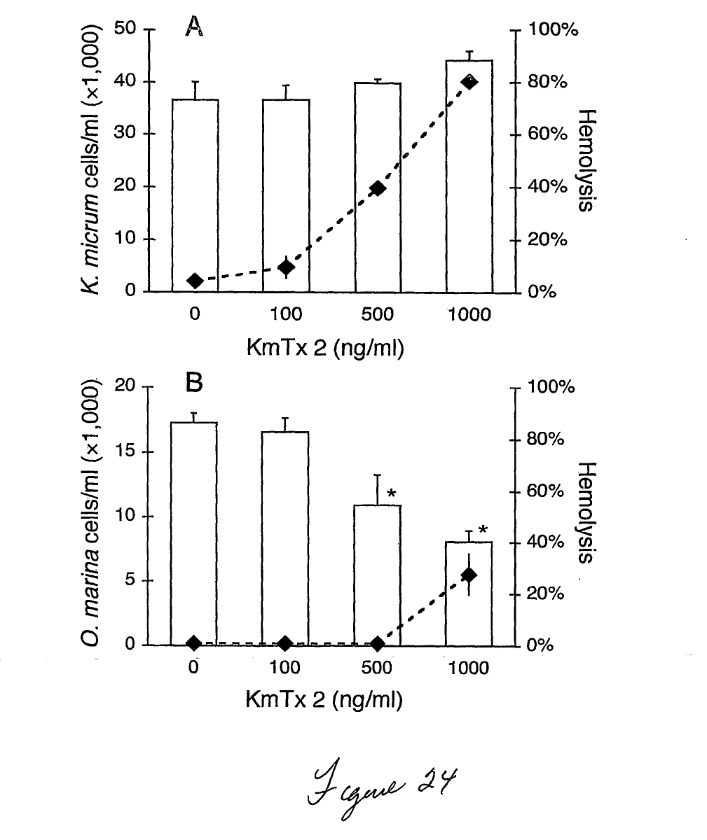

To assess the effects of KmTx 2 on additional eukaryotic cell types, growth inhibition and cytotoxicity assays were performed, respectively, on model yeast and dinoflagellate species. Aspergillis niger was chosen as a representative species of filamentous fungi, while Candida albicans was chosen as a representative species of yeast. To test the hypothesis that KmTx 2 functions as an anti-grazing compound, Oxyhhris marina, a co-occurring, similarly sized, potential grazer, as well as a KmTx 2 producing South Carolina K. micrum isolate were exposed to a range of KmTx 2 concentrations. Minimal inhibitory concentrations (MIC) of 8 and 16 μg/ml, respectively, were found for A. niger and C. albicans for exposure to KmTx 2. Results did not change between 24 and 96 hours. Amphotricin B, a common anti-fungal antibiotic was used as a positive control in the fugal growth inhibition assays. For amphotericin B, no growth was observed in either species at any concentration after 24 hours, but trailing growth resulted in MIC values of 0.5 and 0.125 μg/ml, respectively, for A. niger and C. albicans after 96 hours. Using probit analysis, hemolytic LC₅₀ values for KmTx 2 and amphotericin B were calculated to be 0.368 μg/ml (range: 0.190-0.605) and 3.759 μg/ml (range: 2.067-7.858), respectively. Incubation of KmTx 2 with a Chesapeake Bay isolate of O. marina and a South Carolina isolate of K. micrum (CCMP 2282) resulted in significant (P<0.05) cell lysis to O. marina only in 0.5 and 1 μg/ml treatments at 1 hour (FIG. 24). Furthermore, hemolytic activity remaining in solution after 1 hour was reduced at all KmTx 2 concentrations in O. marina exposed cultures compared to K. micrum exposed cultures. In the 1 μg/ml KmTx 2 treatment, O. marina cell densities were reduced by >90% after 24 hours, while K. micrum cultures continued to divide (data not shown).

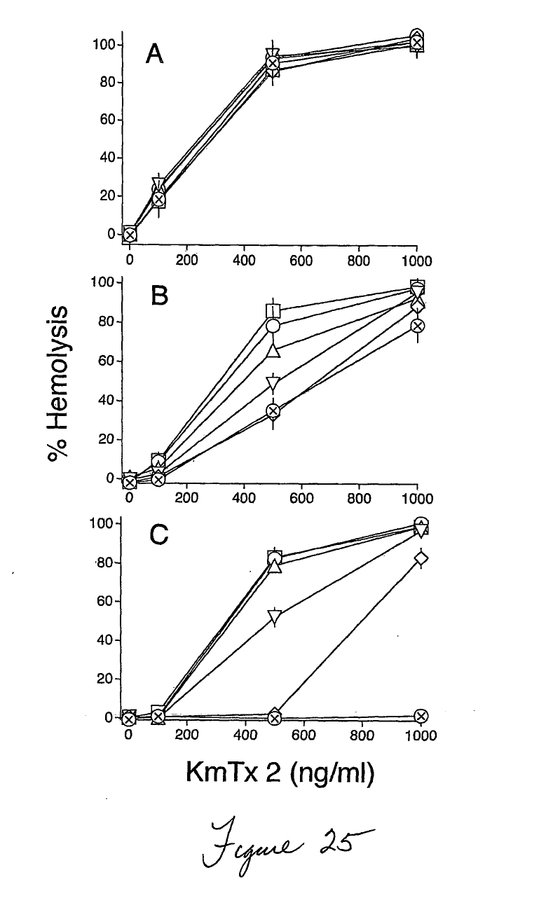

For several cytolytic pore-forming compounds, such as the polyene antibiotics and certain bacterial protein toxins, membrane sterols play a critical role in toxicity. For the polyene antibiotic amphotericin B, binding to target membranes occurs whether sterols are present or not, but permeability leading to cell lysis is only induced when membrane sterols are present. Additional examples of the importance of membrane sterols in cytolysin activity include the amphidinols, potent hemolytic and anti-fungal polyhydroxy-polyenes produced by the dinoflagellate Amphidinium klebsii, whose activity is enhanced in liposomes containing cholesterol, and prymnesins, potent ichthyotoxic and hemolytic polyketides produced by the prymnesiophyte Pryminesium parvum, whose activity is inhibited through co-incubation with cholesterol, ergosterol, and phosphotidylcholine. In this study, the hemolytic activity of KmTx 2 was inhibited by co-incubation with both cholesterol and ergosterol but was not inhibited when incubated with its own major sterol, gymnodinosterol (FIG. 25). In addition, no inhibition was observed upon incubation with either synthetic dipalmitoyl (16:0), or natural brain phosphotidylcholine (data not shown). This data suggests that KmTx 2 will competitively associate with free cholesterol or ergosterol over fish erythrocyte membranes, thereby inhibiting hemolysis, while it will not competitively associate with other common membrane lipoproteins or its own major sterol.

Both cholesterol and ergosterol began to cause inhibition of hemolytic activity in the range of 10-100 nM, but for ergosterol this inhibition plateaued ca. 1 μM whereas with cholesterol, inhibition continued until complete inhibition was reached at levels ca. 10 μM (FIG. 25). In this study, anti-fungal activity of KmTx 2 was 10-100 fold lower than for amphotericin B, depending on species tested. In addition, hemolytic activity towards rainbow trout erythrocytes was ten fold higher for KmTx 2 compared to amphotericin B. All of our data suggest that KmTx 2 has higher activity towards cholesterol containing membranes than ergosterol containing membranes.

In this study, KmTx 2 was toxic towards the co-occurring heterotrophic dinoflagellate, and potential grazer, Oxyrrhis marina while it had no effect on cultures of K. micrum. Furthermore, hemolytic activity remaining in solution after the one-hour incubation period was significantly reduced in O. marina cultures compared to K. micrum cultures suggesting that KmTx 2 will partition into O. marina membranes but not K. micrum membranes.

In summery, KmTx 2, the main toxin from K. micrum populations from North Carolina, South Carolina, and Florida, appears to function by permeabilizing plasma membranes to a range of ions resulting in cell destruction through colloid osmotic lysis. This activity can be inhibited through co-incubation with the membrane sterols cholesterol and ergosterol, but hemolytic and anti-fungal assays suggest that this activity is higher in membranes containing cholesterol. KmTx 2 appears to partition into the membrane of a cholesterol-containing potential grazer, resulting in cell lysis, while it will not partition into it own membrane. This suggests that the natural role of these compounds may be to function as anti-grazing agents. The unusual sterol composition of K. micrum's own membranes appear to play a role in the protection of this organism from its own toxins. Finally, KmTx 2 was toxic towards zebrafish within the range of toxin concentrations found present during fish kills. Sublethal exposure to KmTx 2 resulted in extensive damage to gill epithelia. This work further solidifies the potential ichthyotoxicity of K. micrum, in contrast to previous US reports, and confirms the associations between high densities of this organism and fish kills that have been observed in temperate estuaries around the world for decades.

Literature Cited

All publications mentioned herein are incorporated herein by reference for all purposes.

Arzul, G., Gentien, P., Bodennec, G., Toularastel, F., Youenou, A., and Crassous, M. P., 1995. Comparison of toxic effects in Gymnodinium cf. nagasakiense polyunsaturated fatty acids. In: Lassus, P., Arzul, G., Erard, E., Gentain, P. and Marcaillou, C. (Eds.), Harmful Marine Algal Blooms. Lavoisier, Intercept Ltd., pp. 395-400.

Arzul, G., Gentien, P., and Crassous, M., 1994. A haemolytic test to assay toxins excreted by the marine dinoflagellate Gyrodinium cf. aureolum. Water Res. 28(4), 961-965.

ASTM, 1992. Standard guide for conducting toxicity tests with fishes, macroinvertebrates, and amphibians - E 729-88a. American Society for Testing and Materials, Philadelphia, pp. 403422.

Bodennec, G., Gentien, P., Parrish, C. C., Arzul, G., Youenou, A., and Crassous, M. P., 1995. Production of suspected lipid phycotoxins by Gymnodinium cf. nagasakiense in batch cultures. In: Lassus, P., Arzul, G., Erard, E., Gentain, P. and Marcaillou, C. (Eds.), Harmful Marine Algal Blooms. Lavoisier, Intercept Ltd., pp. 407-412.

Botsford, L. W., Castilla, J. C., and Peterson, C. H., 1997. The management of fisheries and ecosystems. Science 227(5325), 509-515.

Boyd, C. E., 1990. Water Quality in Ponds for Aquaculture. Birmingham Publishing Co., Birmingham, Ala.

Boyd, C. E., 1999. Aquaculture sustainability and environmental issues. World Aquaculture 30(2), 10-13.

Braarud, T. 1957. A red water organism from Walvis Bay (Gymnodinium galatheanum n. sp.). Galathea Report. 1:137-138.

Burkholder, J. M., and Glasgow, H. B., Jr., 1997. Pfiesteria piscicida and other Pfiesteria-like dinoflagellates: Behavior, impacts, and environmental controls. Limnol. Oceanogr. 42(5), 1052-1075.

Burkholder, J. M., Glasgow, H. B., Deamer-Melia, N. J., Springer, J, Parrow, M. W., Zhang, C., Cancellieri, P. J., 2001. Species of the toxic Pfiesteria complex, and the importance of functional type in data interpretation. Environ. Health Perspec. (Supp.) 109(5), 667-679

Burkholder, J. M., Glasgow, H. B., Jr, Lewitus, A., Parrow, M., Zheng, C., Cancellieri, P., and Deamer-Melia, N., 2000. Functional type (toxicity status) controls Pfiesteria response to nutrients and algal prey versus fish prey. Symposium on Harmful Marine Algae in the U.S., Woods Hole, Mass., pp. 24.

Burkholder, J. M., Noga, E. J., Hobbs, C. W., Glasgow, H. B., Jr., and Smith, S. A., 1992. New “phantom” dinoflagellate is the causitive agent of major estuarine fish kills. Nature 358, 407-410. Daugbjerg, N., Hansen, G., Larsen, J., Moestrup, O., 2000. Phylogeny of some of the major genera of dinoflagellates based on ultrastructure and partial LSU rDNA sequence data, including the erection of three new genera of unarmoured dinoflagellates. Phycologia 39(4), 302-317.

Deeds, J. R., Terlizzi, D. E., Stoecker, D. K., Way, R., and Place, A. R., 2000. Relationship between octadecapentaenoic acid, algicidal treatments, and toxicity in two bloom forming dinoflagellates, Karlodinium micrum and Prorocentrum minimum, found in the Chesapeake Bay, Md. Symposium on Harmful Marine Algae in the U.S., Woods Hole, Mass., pp. 106.

Deeds, J. R., Terlizzi, D. E., Adolf, J. E., Stoecker, D. K., and Place, A. R. (2002) Toxic activity from cultures of Karlodinium micrum (=Gyrodinium galatheanum) (Dinophyceae)—A dinoflagellate associated with fish mortalities in an estuarine aquaculture facility. Harmful Algae 1: 169-189.

D'Elia, C. F., Steudler, P. A., and Corwin, N. 1977. Determination and total nitrogen in aqueous samples using persulfate digestion. Limnol. Oceanogr. 22:760-764.

Edvardsen, B., Moy, F., and Paasche, E., 1990. Hemolytic activity in extracts of Chrysochromulina polylepis grown at different levels of selenite and phosphate. In: Graneli, E. (Ed.), Toxic Marine Phytoplankton. Elsevier Science Publishing Co., Inc., Amsterdam, pp. 284-2⁸9.

Edvardsen, B., and Paasche, E., 1998. Bloom dynamics and physiology of Prymnesium and Chrysochromulina. In: Anderson, D. M., Cembella, A. D. and Hallegraeff, G. M. (Eds.), Physiological Ecology of Harmful Algal Blooms. Springer-Verlag, Berlin Heidelberg, pp. 193-208.

Environmental Protection Agency, U. S., 1999. Alternative Disinfectants and Oxidants Guidance Manual—EPA 815-R-99-014. United States Environmental Protection Agency Office of Water. pp. 5-1-5-15.

Fairey, E. R., Edmunds, J. S., Deamer-Melia, N. J., Glasgow, H., Jr., Johnson, F. M., Moeller, P. R., Burkholder, J. M., and Ramsdell, J. S., 1999. Reporter gene assay for fish-killing activity produced by Pfiesteria piscicida. Environ. Health Perspect. 170(9), 711-714.

Fossat, B., Porthe-Nibelle, J., Sola, F., Masoni, A., Gentien, P., and Bodennec, G., 1999. Toxicity of fatty acid 18:5n3 from Gymnodinium cf. mikimotoi: II. Intracellular pH and K+ uptake in isolated trout hepatocytes. J. Appl. Toxicol. 19, 275-278.

Glasgow, H. B., Jr., Lewitus, A. J., and Burkholder, J. M., 1998. Feeding behavior of the ichthyotoxic dinoflagellate, Pfiesteria piscicida, on amino acids, algal prey, and fish vs. mammalian erythrocytes. In. Reguera, B., Blanco, J., Fernandez, M. L., and Wyatt, T. (Eds.), Proceedings, VIIIth International Conference on Harmful Algal Blooms. Xunta de Galicia and Intergovernmental Oceanographic Commission of UNESCO, Paris.

Glasgow, H. B. Jr., Burkholder, J. M., Mallin, M. A., Deamer-Melia, N: J., and Reed, R. E. 2001. Field ecology of toxic Pfiesteria complex species and a conservative analysis of their role in estuarine fish kills. Env. Hlth. Persp. (suppl. 5) 109:715-730.

Glibert, P. M. and Terlizzi, D. E. 1999. Cooccurrence of elevated urea levels and dinoflagellate blooms in temperate estuarine aquaculture ponds. Appl. Environ. Microbiol. 65 (12): 5594-5596.

Glibert, P. M., Magnien, R., Lomas, M. W., Alexander, J., Fan, C., Haramoto, E., Trice, M., and Kana, T. M. 2001. Harmful algal blooms in the Chesapeake and coastal bays of Maryland, USA: Comparison of 1997, 1998, and 1999 events. Estuaries 24:875-883.

Guillard, R. R. L. 1975. Culture of phytoplankton for feeding marine invertebrates. In Smith, W. L. and Chaney, M. H. (Eds.), Culture of Marine Invertebrate Animals. Plenum Press, New York. pp. 29-60.

Guo, M., Harrison, P. J., and Taylor, F. J. R., 1996. Fish kills related to Pryminesium parvum N. Carter (Haptophyta) in the People's Republic of China. J. Appl. Phycol. 8, 111-11. Hallegraeff, G. M., 1993. A review of harmful algal blooms and their apparent global increase. Phycologia 32(2), 79-99.

Johansson, N., and Graneli, E., 1999. Cell density, chemical composition and toxicity of Chrysochromulina polylepis (Haptophyta) in relation to different N:P supply ratios. Mar. Biol. 135, 209-217.

Jones, K. J., Ayres, P., Bullock, A. M., Roberts, R. J., and Tett, P., 1982. A red tide of Gyrodinium auroelum in sea lochs of the Firth of Clyde and associated mortality of pond-reared salmon. J. Mar. Biol. Assoc. U.K. 62, 771-782.

Kempton, J. W., Lewitus, A. J., Deeds, J. R., Law, J. M. & Place, A. R. Toxicity of Karlodinium micrum (Dinophyceae) associated with a fish kill in a South Carolina brackish retention pond. Harmful Algae 1, 233-241 (2002).

Khan, S., Arakawa, O., and Onoue, Y., 1997. Neurotoxins in a toxic red tide of Heterosigma akashiwo (Raphidophyceae) in Kagoshima Bay, Japan. Aquac. Res. 28, 9-14.

Larsen, J. and Moestrup, O. 1989. Guide to toxic and potentially toxic marine algae. Institut for Sporeplanter, University of Copenhagen. Fish Inspection Service, Ministry of Fisheries, Copenhagen, Denmark, pp. 33-36.

Lewitus, A. J., Burkholder, J. M., Glasgow Jr., H. B., Glibert, P. M., Willis, B. M., Hayes, K. C. and Burke, M. K. 1999. Mixotrophy and nutrient uptake by Pfiesteria piscicida (Dinophyceae). J. Phycol. 35:1430-1437.

Lewitus, A. J. and Holland, A. F. 2002. Initial results from a multi-institutional collaboration to monitor harmful algal blooms in South Carolina. In: Proceedings of the EMAP Symposium 2001: Coastal Monitoring Through Partnership; Environmental Monitoring and Assessment (special issue), pp. 365-375.

Lewitus, A. J., Hayes, K. C., Willis, B. M., Burkholder, J. M., Glasgow, Jr. H. B., Holland, A. F., Maier, P., Rublee, P. A., and Magnien, R. 2002. Low abundance of the dinoflagellates, Pfiesteria piscicida, P. shumwayae, and Cryptoperidiniopsis spp. in South Carolina estuaries: Relevance as reference sites to areas impacted by Pfiesteria toxic events. Estuaries 25.

Li, A., Stoecker, D. K., Coats, D. W., and Adams, E. J. 1996. Ingestion of fluorescently-labeled and phycoerythrin-containing prey by photosynthetic dinoflagellates. Aquat. Microb. Ecol. 10: 139-147.

Li, A., Stoecker, D. K., and Adolf, J. E., 1999. Feeding, pigmentation, photosynthesis and growth of the mixotrophic dinoflagellate Karlodinium micrum. Aquat. Microb. Ecol. 19, 163-176.

Li, A., Stoecker, D. K., and Coats, D. W. 2000. Mixotrophy in Gyrodinium galatheanum (Dinophyceae): grazing responses to light intensity and inorganic nutrients. J. Phycol. 36:33-45.

Li, A., Stoecker, D. K., and Coats, D. W., 2000. Spatial and temporal aspects of Gyrodinium galatheanum in Chesapeake Bay: distribution and mixotrophy. J. Plankton. Res. 22(11), 2105-2124.

Marshall, H. G., 1999. Pfiesteria piscicida and dinoflagellates similar to Pfiesteria. VA. J. Science 50(4), 281-285.

Masser, M. P., 2000. Aquatic vegetation control. In: R. R. Stickney (Ed.), Encyclopedia of Aquaculture. John Wiley and Sons Inc., New York, pp. 51-61.

Millie D. F., Kirkpatick, G. J., and Vinyard, B. T. 1995. Relating photosynthetic pigments in vivo optical density spectra to irradiance for the Florida red-tide dinoflagellate Gymnodinium breve. Mar. Ecol. Prog. Ser. 120:65-75.

Nielsen, M. V. 1993. Toxic effect of the marine dinoflagellate Gymnodinium galatheanum on juvenile cod Gadus morhua. Mar. Ecol. Prog. Ser. 95:273-277.

Nielsen, M. V. 1996. Chemical composition of the toxic dinoflagellate Gymnodinium galatheanum in relation to irradiance, temperature and salinity. Mar. Ecol. Prog. Ser. 136:205-211.

Nielsen, M. V. and Stromgren, T. 1991. Shell growth response of mussels (Mytilus edulis) exposed to toxic microalgae. Mar. Biol. 108:263-267.

Noga, E. J., 2000. Fish Disease: Diagnosis and Treatment. Iowa State University Press, Ames, Iowa.

Parrish, C. C., Bodennec, G., and Gentien, P., 1998. Haemolytic glycolipids from Gymnodinium species. Phytochemistry 47(5), 783-787.

Parrish, C. C., Bodennec, G., Sebedio, J.-L., and Gentien, P., 1993. Intra- and extracellular lipids in cultures of the toxic dinoflagellate, Gyrodinium aureolum. Phytochemistry 32(2), 291-295.

Pieterse, F. and Van Der Post, D. C., 1967. The Pilchard of South West Africa (Sardinops ocellata) Oceanographic conditions associated with red-tides and fish mortalities in the Walvis Bay region. Investigational Report No. 14, 8-27.

Reardon, I. S., and Harrell, R., M., 1990. Acute toxicity of formalin and copper sulfate to striped bass fingerlings held in varying salinities. Aquaculture 87, 255-270.

Roberts, R. J., Bullock, A. M., Turner, M., Jones, K., and Tett, P., 1983. Mortalities of Salmo gairdneri exposed to cultures of Gyrodinium aureolum. J. Mar. Biol. Assoc. U.K. 63, 741-743.

Schaefer, E. F. 1997. A DNA assay to detect the toxic dinoflagellate Pfiesteria piscicida, and the application of a PCR based probe. M.S. Thesis, University of North Carolina at Greensboro.

Smayda, T. J. 1997. Harmful algal blooms: their ecophysiology and general relevance to phytoplankton blooms in the sea. Limnol. Oceanogr. 42:1137-1153.

Smayda, T. J. 1998. Ecophysiology and bloom dynamics of Heterosigma akashiwo (Raphidophyceae). In: Anderson, D. M., Cembella, A. D., and Hallegraeff, G. M. (Eds.) Physiological Ecology of Harmful Algal Blooms. Springer-Verlag, Berlin, pp. 113-131.

Sola, F., Masoni, A., Fossat, B., Porthe-Nibelle, J., Gentien, P., and Bodennec, G., 1999. Toxicity of fatty acid 18:5n3 from Gymnodinium cf. mikimotoi: I. Morphological and biochemical aspects on Dicentrarchus labrax gills and intestines. J. Appl. Toxicol. 19, 279-284.

Steidinger, K. A., Burkholder, J. M., Glasgow, H. B. J., Hobbs, C. W., Garrett, J. K., Truby, E. W., Noga, E. J., and Smith, S. A., 1996. Pfiesteria piscicida gen. et sp. nov. (Pfiesteriaceae fam. nov.), A new toxic dinoflagellate with a complex life cycle and behavior. J. Phycol. 32, 157-164.

Tengs, T., Bowers, H. A., Ziman, A. P., Stoecker, D. K., and Oldach, D. W. 2001. Genetic polymorphism in Gymnodinium galatheanum chloroplast DNA sequences and development of a molecular detection assay. Mol. Ecol. 10(2):515-523.

Terlizzi, D. E., Stoecker, D. K., Glibert, P. M. 2000. Gyrodinium galatheanum: a threat to estuarine aquaculture waters. In: Responsible Aquaculture in the New Millenium. Abstracts of Contributions Presented at the International Conference AQUA 2000, Nice, France, May 2-6, 2000 (Eds. Flos R, Cressell L), European Aquaculture Society Special Publication 28, Ostende, Belgium. pp. 700.

Tucker, C. S., 1987. Acute toxicity of potassium permanganate to channel catfish fingerlings. Aquaculture 60, 93-98.

Tucker, C. S., 1989. Method for estimating potassium permanganate disease treatment rates for channel catfish in ponds. Prog. Fish. Cult. 51, 24-26.

Tucker, C. S., and Boyd, C. E., 1977. Relationships between potassium permanganate treatment and water quality. T. Am. Fish. Soc. 106(5), 481488.

Ulitzer, S., and Shilo, M., 1966. Mode of action of Prymnesium parvum ichthyotoxin. J. Protozool. 13(2), 332-336.

Ulitzer, S., and Shilo, M., 1970. Procedure for purification and separation of Prymnesium parvum toxins. Biochim. Biophys. Acta 201, 350-363.

Yongmanitchai, W. & Ward, O. P. Separation of lipid classes from Phaeodactylum tricornutum using silica cartridges. Phytochemistry 31, 3405-3408 (1992).

Xi, D., Kurtz, D. T., and Ramsdell, J. S., 1996. Maitotoxin-elevated cytosolic free calcium in GH4C1 rat pituitary cells nimodiphine-sensitive and -insensitive mechanisms. Biochem. Pharmacol. 51(6) 759-769.

Yasumoto, T., Underdal, B., Aune, T., Hormazabal, V., Skulberg, O. M., and Oshima, Y., 1990. Screening for hemolytic and ichthyotoxic components of Chrysochromulina polylepis and Gyrodinium aureolum from Norwegian coastal waters. In: Granell, E., (Ed.), Toxic Marine Phytoplankton. Elsevier Science Publishing Co., Inc., Amsterdam, pp. 436-440. Young, R. C., McLaren, M., and Ramsdell, J. S., 1995. Maitotoxin increases voltage independent chloride and sodium currents in GH4C1 rat pituitary cells. Nat. Toxins 3(6), 419-427.

Yongmanitchai, W., and Ward, O. P., 1992. Separation of lipid classes from Phaeodactylum tricornutum using silica cartridges. Phytochemistry 31(10), 3405-3408.

1. An isolated toxin from Karlodinium micrum.

2. The isolated toxin according to claim 1 comprising a member selected from the group consisting of KmTx 1, KmTx 2, KmTx 3, KmTx 4, KmTx 5 and KmTx 6.

3. The isolated toxin according to claim 1 comprising KmTx 1, wherein the KmTx 1 toxin is eluted at about 22-24 minutes of reversed phase HPLC elution of Karlodinium micrum.

4. The isolated toxin according to claim 1 comprising KmTx 3, wherein the KmTx 3 toxin is eluted at about 16-18 minutes of reversed phase HPLC elution of Karlodinium micrum.

5. The isolated toxin according to claim 1 comprising KmTx 1, wherein the KmTx 1 toxin is eluted at about 22 to 24 minutes of reversed phase HPLC fractions of a concentrated 80% MeOH tC₁₈ elution of Karlodinium micrum.

6. The isolated toxin according to claim 1 comprising KmTx 3, wherein the KmTx 3 toxin is eluted at about 16-18 minutes of reversed phase HPLC fractions of a concentrated 80% MeOH tC₁₈ elution of Karlodinium micrum.

7. The isolated toxin according to claim 1 comprising a molecular mass of 1362 daltons.

8. The isolated toxin according to claim 1 comprising a molecular mass of 1344 daltons.

9. A method of producing a karlotoxin comprising the steps of: a) culturing Karlodinium micrum in a medium suitable for production of toxin; and b) isolating the toxin.

10. The method according to claim 9 comprising a member selected from the group consisting of KmTx 1, KmTx 2, KmTx 3, KmTx 4, KmTx 5 and KmTx 6.

11. The method according to claim 9, wherein the toxin is isolated by separation on a HPLC column.

12-19. (canceled)

20. A method of inhibiting a Karlodinium micrum toxin comprising contacting an antibody which specifically binds said toxin.

21. The method of claim 20, wherein the toxin is the toxin comprises a member selected from the group consisting of KmTx 1, KmTx 2, KmTx 3, KmTx 4, KmTx 5 and KmTx 6.

22. The method of claim 20, wherein the toxin is the toxin comprises KmTx 1 and is eluted at about 22-24 minutes of reversed phase HPLC elution of Karlodinium micrum.

23. The method of claim 20, wherein the toxin is the toxin comprises KmTx 3 and is eluted at about 17-18 minutes of reversed phase HPLC elution of Karlodinium micrum.

24. An immunoconjugate comprising a Karlodinium micrum toxin linked to an antibody.

25. The immunoconjugate of claim 24, wherein the toxin comprises a member selected from the group consisting of KmTx 1, KmTx 2, KmTx 3, KmTx 4, KmTx 5 and KmTx 6.

26. The immunoconjugate of claim 25, wherein the antibody is an anti-tumor antibody.

27.-28. (canceled)

29. A composition comprising a Karlodinium micrum toxin.

30. The composition according to claim 29, wherein the toxin comprises a member selected from the group consisting of KmTx 1, KmTx 2, KmTx 3, KmTx 4, KmTx 5 and KmTx 6.

31. The composition according to claim 29, wherein the toxin comprises KmTx 1 and is eluted at about 22-24 minutes of reversed phase HPLC elution of Karlodinium micrum.

32. The composition according to claim 29, wherein the toxin comprises KmTx 3 and is eluted at about 17-18 minutes of reversed phase HPLC elution of Karlodinium micrum.

33. A method of treating blooms in an aqueous medium caused by K. micrum to reduce mortality rate of fish exposed to the treatment, the method comprising:
introducing an algicidal composition in an effective amount to reduce the level of K. micrum in the aqueous medium, wherein the algicidal composition comprises potassium permanganate and does not include copper sulfate.

34.-36. (canceled)
